SEQUENCE LISTING (8 sequences)

SEQ ID NO: 1 - RNA, 19 nt, Artificial
  Oligomer
gugccaaccugaugcagcu

SEQ ID NO: 2 - RNA, 19 nt, Artificial
  Oligomer
agcugcaucagguuggcac

SEQ ID NO: 3 - RNA, 19 nt, Artificial
  Oligomer
gagaauauuucacccuuca

SEQ ID NO: 4 - RNA, 19 nt, Artificial
  Oligomer
ugaagggugaaauauucuc

SEQ ID NO: 5 - RNA, 19 nt, Artificial
  Oligomer
ucccguuuguagaugacaa

SEQ ID NO: 6 - RNA, 19 nt, Artificial
  Oligomer
uugucaucuacaaacggga

SEQ ID NO: 7 - RNA, 21 nt, Artificial
  Oligomer
ggcuacguccaggagcgcacc

SEQ ID NO: 8 - RNA, 21 nt, Artificial
  Oligomer
ggugcgcuccuggacguagcc

(19) United States
(12) Patent Application Publication
(10) Pub. No.: US 2010/0215588 A1
(43) Pub. Date: Aug. 26, 2010

(54) THERAPEUTIC DELIVERY OF INHIBITORY NUCLEIC ACID MOLECULES TO THE RESPIRATORY SYSTEM

(76) Inventor: Rami Skaliter, Nes Ziona (IL)

Correspondence Address: COOPER & DUNHAM, LLP, 30 Rockefeller Plaza, 20th Floor, NEW YORK, NY, 10112, US

(21) Appl. No.: 12/451,140

(22) PCT Filed: Apr. 27, 2008
(86) PCT No.: PCT/IL2008/000522

§ 371 Date: Apr. 15, 2010

Related U.S. Application Data
(60) Provisional application No. 60/926,559, filed on Apr. 26, 2007.

(51) Int. Cl. A61K 9/12; A61P 11/00

(52) U.S. Cl. 424/45

(57) ABSTRACT

The present invention relates to methods of treating respiratory disorders of all types, including pulmonary disorders, by delivering inhibitory nucleic acid molecules directly to the respiratory system. siRNAs or other nucleic acids are delivered to the lung/respiratory system for the treatment of disease.

This application claims priority of U.S. Provisional patent application No. 60/926,559, filed 26 Apr. 2007, which is hereby incorporated by reference in its entirety.

Throughout this application various patent and scientific publications are cited. The disclosures for these publications in their entireties are hereby incorporated by reference into this application to more fully describe the state of the art to which this invention pertains.

### FIELD OF THE INVENTION

The present invention relates to methods of treating respiratory disorders of all types (including pulmonary disorders), by delivering inhibitory nucleic acid molecules directly to the respiratory system.

### BACKGROUND OF THE INVENTION

Lung diseases comprise a spectrum of manifestations and etiologies, and may be particularly difficult to treat with systemic administration of potential therapeutics. Over 150 diseases of the interstitium, the tissue between the alveoli, have been identified, including many types of fibrosis. Other lung diseases include disorders of gas exchange, disorders of blood circulation, disorders of the airways, and disorders of the pleura. Lung cancers include both primary lung cancers and metastases from primary cancers of various other organs or tissues. Infectious diseases of the lung include viral, bacterial, and fungal infectious agents. A number of general methods have been described for delivering medically important molecules, including small molecules, nucleic acids, and/or protein or peptide compositions, in an effort to improve bioavailability and/or to target delivery to particular locations within the body. Such methods include the use of prodrugs, encapsulation into liposomes or other particles, and co-administration in uptake enhancing formulations (for review see, e.g., Critical Reviews in Therapeutic Drug Carrier Systems, Stephen D. Bruck, ed., CRC Press, 1991).

### Chronic Obstructive Pulmonary Disease (COPD)

Chronic obstructive pulmonary disease, (COPD), affects more than 16 million Americans and is the fourth highest cause of death in the United States. Cigarette smoking causes most occurrences of the debilitating disease but other environmental factors cannot be excluded (Petty T L. 2003. Clin. Cornerstone, 5-10).

Pulmonary emphysema is a major manifestation of COPD. Permanent destruction of peripheral air spaces, distal to terminal bronchioles, is the hallmark of emphysema (Tuder R M, et al.

Am J Respir Cell Mol Biol, 29:88-97; 2003). Emphysema is also characterized by accumulation of inflammatory cells such as macrophages and neutrophils in bronchioles and alveolar structures (Petty, 2003).

The pathogenesis of emphysema is complex and multifactorial. In humans, a deficiency of inhibitors of proteases produced by inflammatory cells, such as alpha1-antitrypsin, has been shown to contribute to protease/antiprotease imbalance, thereby favoring destruction of alveolar extracellular matrix in cigarette-smoke (CS) induced emphysema (Eriksson, S. 1964. Acta Med Scand 175:197-205. Joos, L., Pare, P. D., and Sandford, A. J. 2002. Swiss Med Wkly 132:27-37). Matrix metalloproteinases (MMPs) play a central role in experimental emphysema, as documented by resistance of macrophage metalloelastase knockout mice against emphysema caused by chronic inhalation of CS (Hautamaki, et al: Science 277:2002-2004). Moreover, pulmonary overexpression of interleukin-13 in transgenic mice results in MMP- and cathepsin-dependent emphysema (Zheng, T., et al 2000. J Clin Invest 106:1081-1093). Recent works describe involvement of septal cell apoptosis in lung tissue destruction leading to emphysema (Rangasamy T, et al. J. Clin. Invest. 114(9): 1248-1259 (2004); Tuder R M et al. Am J Respir Cell Mol Biol, 29:88-97; 2003; Yokohori N, Aoshiba K, Nagai A, Chest. 2004 February; 125(2):626-32; Aoshiba K, Yokohori N, Nagai A., Am J Respir Cell Mol. Biol. 2003 May; 28(5): 555-62).

Among the mechanisms that underlie both pathways of lung destruction in emphysema, excessive formation of reactive oxygen species (ROS) should be first of all mentioned. It is well established that prooxidant/antioxidant imbalance exists in the blood and in the lung tissue of smokers (Hulea S A, et al: J Environ Pathol Toxicol Oncol. 1995; 14(3-4):173-80; Rahman I, MacNee W. Am J. Physiol. 1999 December; 277(6 Pt 1):L1067-88; MacNee W. Chest. 2000 May; 117(5 Suppl 1):303S-17S; Marwick J A, Kirkham P, Gilmour P S, Donaldson K, MacNEE W, Rahman I. Ann NY Acad. Sci. 2002 November; 973:278-83; Aoshiba K, Koinuma M, Yokohori N, Nagai A. Inhal Toxicol. 2003 September; 15(10):1029-38; Dekhuij zen P N. Eur Respir J. 2004 April; 23(4):629-36; Tuder R M, Then L, Cho C Y, Taraseviciene-Stewart L, Kasahara Y, Salvemini D, Voelkel N F, and Flores S C. Am J Respir Cell Mol Biol, 29:88-97; 2003). After one hour exposure of mice to CS, there is a dramatic increase of 8-hydroxy-2′-deoxyguanosine (8-OHdG) in the alveolar epithelial cells, particularly of type II (see Inhal Toxicol. 2003 September; 15(10):1029-38. above).

Overproduced reactive oxygen species are known for their cytotoxic activity, which stems from a direct DNA damaging effect and from the activation of apoptotic signal transduction pathways (Takahashi A, Masuda A, Sun M, Centonze V E, Herman B. Brain Res Bull. 2004 Feb. 15; 62(6):497-504; Taniyama Y, Griendling K K. Hypertension. 2003 December; 42(6):1075-81. Epub 2003 Oct. 27; Higuchi Y. Biochem Pharmacol. 2003 Oct. 15; 66(8):1527-35; Punj V, Chakrabarty A M. Cell Microbiol. 2003 April; 5(4):225-31; Ueda S, Masutani H, Nakamura H, Tanaka T, Ueno M, Yodoi J. 2002 June; 4(3):405-14).

ROS's are not only cytotoxic per se but are also proinflammatory stimuli, being prominent activators of redox-sensitive transcription factors NFkB and AP-1 (reviewed in Rahman I. Curr Drug Targets Inflamm Allergy. 2002 September; 1(3):291-315). Both transcription factors are, in turn, strongly implicated in stimulation of transcription of proinflammatory cytokines (reviewed in Renard P, Raes M. Cell Biol Toxicol. 1999; 15(6):341-4; Lentsch A B, Ward P A. Arch Immunol Ther Exp (Warsz). 2000; 48(2):59-63) and matrix degrading proteinases (Andela V B, Gordon A H, Zotalis G, Rosier R N, Goater J J, Lewis G D, Schwarz E M, Puzas J E, O'Keefe R J. NFkappaB: Clin Orthop. 2003 October; (415 Suppl):S75-85; Fleenor D L, Pang I H, Clark A F. Invest Opthalmol Vis Sci. 2003 August; 44(8):3494-501; Ruhul Amin A R, Senga T, Oo M L, Thant A A, Hamaguchi M. Genes Cells. 2003 June; 8(6):515-23). Proinflammatory cytokines, in turn, serve as attractors of inflammatory cells that also secrete matrix degrading enzymes, cytokines and reactive oxygen species. Thus, it appears that a pathogenic factor, like e.g. CS, triggers a pathological network where reactive oxygen species act as major mediators of lung destruction.

Both reactive oxygen species (ROS) from inhaled cigarette smoke and also those endogenously formed by inflammatory cells contribute to an increased intrapulmonary oxidant burden.

One additional pathogenic factor with regards to COPD pathogenesis is the observed decreased expression of VEGF and VEGFRII in lungs of emphysematous patients (Yasunori Kasahara, Rubin M. Tuder, Carlyne D. Cool, David A. Lynch, Sonia C. Flores, and Norbert F. Voelkel. Am J Respir Crit. Care Med Vol 163. pp 737-744, 2001). Moreover, inhibition of VEGF signaling using chemical VEGFR inhibitor leads to alveolar septal endothelial and then to epithelial cell apoptosis, probably due to disruption of intimate structural/functional connection of both types of cells within alveoli (Yasunori Kasahara, Rubin M. Tuder, Laimute Taraseviciene-Stewart, Timothy D. Le Cras, Steven Abman, Peter K. Hirth, Johannes Waltenberger, and Norbert F. Voelkel. J. Clin. Invest. 106:1311-1319 (2000); Voelkel N F, Cool C D. Eur Respir J. Suppl. 2003 November; 46:28s-32s).

### Lung Cancer

Lung cancer is a cancer that forms in tissues of the lung, usually in the cells lining air passages.

The two main types are small cell lung cancer and non-small cell lung cancer. These types are diagnosed based on the morphology of the cells under a microscope. It is the most lethal of all cancers worldwide, responsible for up to 3 million deaths annually. In non-small cell lung cancer (NSCLC), results of standard treatment are poor except for the most localized cancers. Surgery is the most potentially curative therapeutic option for this disease; radiation therapy can produce a cure in a small number of patients and can provide palliation in most patients. Adjuvant chemotherapy may provide an additional benefit to patients with resected NSCLC. In advanced-stage disease, chemotherapy offers modest improvements in median survival, though overall survival is poor. Chemotherapy has produced short-term improvement in disease-related symptoms.

siRNAs and RNA Interference

The present invention relates generally to compounds which down-regulate expression of two or more genes, and particularly to novel small interfering RNAs (siRNAs), and to the use of these novel siRNAs in the treatment of various diseases and medical conditions.

The present invention provides methods and compositions for inhibiting expression of the target genes in vivo. In general, the method includes administering oligoribonucleotides, such as small interfering RNAs (i.e., siRNAs) that are targeted to two or more particular mRNA and hybridize to, or interact with, it under biological conditions (within the cell), or a nucleic acid material that can produce siRNA in a cell, in an amount sufficient to down-regulate expression of two or more target genes by an RNA interference mechanism. Additionally the siRNAs of the invention can be used in vitro as part of a compound screening system to look for small compounds that compete with, or overcome effect of, siRNAs.

RNA interference (RNAi) is a phenomenon involving double-stranded (ds) RNA-dependent gene specific posttranscriptional silencing. Originally, attempts to study this phenomenon and to manipulate mammalian cells experimentally were frustrated by an active, non-specific antiviral defence mechanism which was activated in response to long dsRNA molecules; see Gil et al. 2000, Apoptosis, 5:107-114. Later it was discovered that synthetic duplexes of 21 nucleotide RNAs could mediate gene specific RNAi in mammalian cells, without the stimulation of the generic antiviral defence mechanisms (see Elbashir et al. Nature 2001, 411:494-498 and Caplen et al. Proc Natl Acad Sci 2001, 98:9742-9747). As a result, small interfering RNAs (siRNAs), which are short double-stranded RNAs, have become powerful tools in attempting to understand gene function.

Thus, RNA interference (RNAi) refers to the process of sequence-specific post-transcriptional gene silencing in mammals mediated by small interfering RNAs (siRNAs) (Fire et al, 1998, Nature 391, 806) or microRNAs (miRNAs) (Ambros V. Nature 431:7006 ,350-355(2004); and Bartel D P. Cell. 2004 Jan. 23; 116(2): 281-97). The corresponding process in plants is commonly referred to as specific post-transcriptional gene silencing or RNA silencing and is also referred to as quelling in fungi. An siRNA is a double-stranded RNA molecule which down-regulates or silences (prevents) the expression of a gene/mRNA of its endogenous (cellular) counterpart. RNA interference is based on the ability of dsRNA species to enter a specific protein complex, where it is then targeted to the complementary cellular RNA and specifically degrades it. Thus, the RNA interference response features an endonuclease complex containing an siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir et al 2001, Genes Dev., 15, 188). In more detail, longer dsRNAs are digested into short (17-29 bp) dsRNA fragments (also referred to as short inhibitory RNAs—“siRNAs”) by type III RNAses (DICER, DROSHA, etc., Bernstein et al., Nature, 2001, v. 409, p. 363-6; Lee et al., Nature, 2003, 425, p. 415-9). The RISC protein complex recognizes these fragments and complementary mRNA. The whole process is culminated by endonuclease cleavage of target mRNA (McManus &Sharp, Nature Rev Genet, 2002, v. 3, p. 737-4′7; Paddison &Hannon, Curr Opin Mol. Ther. 2003 June; 5(3): 217-24). For information on these terms and proposed mechanisms, see Bernstein E., Denli A M. Hannon G J: 2001. RNA. I; 7(11): 1509-21; Nishikura K.: 2001 Cell. I 16; 107(4): 415-8 and PCT publication WO 01/36646 (Glover et al).

The selection and synthesis of siRNA corresponding to known genes has been widely reported; see for example Chalk A M, Wahlestedt C, Sonnhammer E L. 2004 Biochem. Biophys. Res. Commun. June. 18; 319(1): 264-74; Sioud M, Leirdal M., 2004, Methods Mol. Biol.; 252:457-69; Levenkova N, Gu Q, Rux J J. 2004, 112; 20(3): 430-2. and Ui-Tei K, Naito Y, Takahashi F, Haraguchi T, Ohki-Hamazaki H, Juni A, Ueda R, Saigo K., Nucleic Acids Res. 2004 I 9; 32(3):936-48.5e also Liu Y, Braasch D A, Nulf C J, Corey D R., Biochemistry, 2004 I 24; 43(7):1921-7. See also PCT publications WO 2004/015107 (Atugen) and WO 02/44321 (Tuschl et al), and also Chiu Y L, Rana T M. RNA 2003 September; 9(9):1034-48 and U.S. Pat. Nos. 5,898,031 and 6,107,094 (Crooke) for production of modified/more stable siRNAs.

Several groups have described the development of DNA-based vectors capable of generating siRNA within cells. The method generally involves transcription of short hairpin RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

Several studies have revealed that siRNA therapeutics are effective in vivo in both mammals and in humans. Bitko et al., have shown that specific siRNA molecules directed against the respiratory syncytial virus (RSV) nucleocapsid N gene are effective in treating mice when administered intranasally (Bitko et al., Nat. Med. 2005, 11(1):50-55). For reviews of therapeutic applications of siRNAs see for example, Barik (Mol. Med. 2005, 83: 764-773); Chakraborty (Current Drug Targets 2007 8(3):469-82) and Dykxhoorn, et al (Gene Therapy 2006, 13, 541-552). Furthermore, a phase I clinical study with short siRNA molecule that targets the VEGFR1 receptor for the treatment of Age-Related Macular Degeneration (AMD) has been conducted in human patients. In studies such siRNA administered by intravitreal (intraocular) injection was found effective and safe in 14 patients tested (Kaiser, Am J. Opthalmol. 2006 142(4):660-8).

Pulmonary administration of therapeutic compositions comprised of low molecular weight drugs, for example, beta-androgenic antagonists to treat asthma, has been observed. Other therapeutic agents that are active in the lungs have been administered systemically and targeted via pulmonary absorption. Direct delivery to the respiratory system, optionally oro-nasal or tracheal delivery, may be considered to be a promising technique for administration of therapeutics for the following reasons: the nose has a large surface area available for drug absorption due to the coverage of the epithelial surface by numerous microvilli, the sub-epithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm.sup.3, porous endothelial basement membrane, and it is easily accessible.

The effectiveness of a pharmaceutical or drug depends on efficient delivery to target cells of interest. Aerosol delivery is non-invasive and has the potential for delivering high concentrations of the therapeutic molecule. Aerosol delivery of nucleic acids to the lungs using viral vectors, polymers, surfactants, or excipients has been described. McDonald et al., describes aerosol delivery of an adenoviral vector encoding the cystic fibrosis transmembrane conductance regulator protein (CFTR) to non-human primates (McDonald, et al., Human Gene Therapy 8:411-422 (1997)). Canonico, et al., describes the in vivo gene transfer of a plasmid containing recombinant human alpha 1-antitrpsin gene and a cytomegalovirus promoter complexed to cationic liposomes to the lungs by aerosol to rabbits (Canonico, et al., Am. J. Respir. Cell Mol. Biol., 10:24-29 (1994)). Stribling et al., describes that the aerosol delivery of a chloramphenicol acetyltransferase reporter gene complexed to a cationic liposome carrier can produce CAT gene expression in mouse lungs (Stribling, et al., Proc. Natl. Acad. Sci. USA 89:11277-11281 (1992)). Massaro, et al., describes delivery of small inhibitory RNA molecules complexed to the lipoprotein pulmonary surfactant, known as surface active material or SAM, to the pulmonary alveoli in mice via liquid deposition into the nasal orifice (Massaro, et al., Am. J. Physiol. Lung Cell Mol. Physiol. 287:L 1066-L1070 (2004)). U.S. patent application No. 2005/0008617 by Chen, et al., describes delivery of RNAi-inducing agents including short-interfering RNA (siRNA), short hairpin RNA (shRNA), and RNAi-inducing vectors complexed with cationic polymers, modified cationic polymers, lipids, and/or surfactants suitable for introduction into the lung. U.S. patent application No. 2003/0157030 by Davis, et al., describes administration of RNAi constructs such as siRNAs or nucleic acids that produce siRNAs complexed with polymers for nasal delivery.

Among the limited number of other reports on siRNA administration in vivo, all use systemic delivery, transfection chemicals, or viral vectors (Hasuwa, et al., FEBS Lett., 532:227-230 (2002); McCaffrey, et al., Nature, 418:38-39 (2002); Reich, et al., Mol. Vis., 9:210-216 (2003); Sorensen, et al., J. Mol. Biol., 327:761-766 (2003); and Zender, et al., Proc. Natl. Acad. Sci. U.S.A., 100:7797-7802 (2003)).

Direct delivery to the inner lung offers the possibility of treatment for conditions such as COPD, bronchitis, asthma, cystic fibrosis, lung cancer, pulmonary fibrosis and acute respiratory distress syndrome, for which current therapy is inadequate. The toxicity associated with the use of systemic delivery of nucleic acids and/or a transfection chemical or viral vector raises concerns for clinical use; in addition, the use of plasmid DNA constructs limits efficacy and delivery of plasmid DNA as a potential therapeutic agent. Thus, the present invention offers the advantages of lowered potential side-effects and increased efficacy.

It is therefore an object of the present invention to provide compositions for intra-lung administration containing nucleic acids without vectors and preferably with little to no polymers, surfactants, or excipients—for the treatment of various pulmonary diseases.

Due to the difficulty in identifying and obtaining regulatory approval for chemical drugs for the treatment of diseases, the delivery modes of the present invention offer an advantage in that they are safe, lack side effects and may be used to deliver various pharmaceutical compositions for treatment of any disease.

### SUMMARY OF THE INVENTION

Methods for intra lung administration are described herein that contain nucleic acids, particularly siRNAs, without viral or plasmid vectors and preferably with little or no polymers, surfactants, or excipients. In one embodiment, the composition for delivery consists essentially of at least one nucleic acid molecule and an aqueous solution. Suitable nucleic acids for intranasal delivery include, but are not limited to, dsDNA, dsRNA, ssDNA, ssRNA, short interfering RNA, micro-RNA, and antisense RNA. In one embodiment, the size range of the nucleic acids is 30 or 23 nucleotides or less in length, although oligonucleotide molecules of between 5 and up to 60 nucleotides can be utilized. In a preferred embodiment the size range of the nucleic acids is between 19 to 23 nucleotides in length.

The compositions are administered to a patient in need of treatment, prophylaxis or diagnosis of at least one symptom or manifestation of a lung disease. In one embodiment, the compositions for oro-nasal and/or intra-lung administration are administered in an effective amount to inhibit gene expression, preferably in the lung. The composition is administered in a dose range of 1 to 10 grams per kilogram (g) of body weight of the subject daily, with upper dosing limit of 0.1 kilogram per kilogram body weight daily. In a preferred embodiment the composition is administered in a dose range of 1 to 5 grams per kilogram of body weight daily. In one embodiment, the composition is administered in an effective amount to inhibit expression of RTP801 in the lung. In another embodiment, the composition is administered in an effective amount to inhibit expression of p53 in the lung. For further information, see PCT patent application publication Nos. WO06/023544A2 and WO06/035434 assigned to the assignee of the present invention. Additional genes the expression of which is preferably inhibited in the inner lung include but are not limited to RTP801, p53, TP53BP2, LRDD, CYBA, ATF3, CASP2, NOX3, HRK, C1QBP, BNIP3, MAPK8, MAPK14, Rac1, GSK3B, P2RX7, TRPM2, PARG, CD38, STEAP4, BMP2, GJA1, TYROBP, CTGF, SPP1, RTN4R, ANXA2, RHOA DUOX1, NRF2, REDD2, NOX4, MYC, PLK1, ESPL1, KEAP1, and SHCl, and particularly DUOX1, TYROBP and CTGF.

Methods for treatment, diagnosis, or prevention of at least one symptom of a lung disease are also described consisting of administration by oro-nasal or oral or nasal delivery to the inner lung of an effective amount of a composition containing a nucleic acid molecule, preferably an siRNA, to alleviate at least one symptom. The composition is typically formulated as an aerosol or other acceptable formulation for oro-nasal administration. In one embodiment, the composition delivered by oro-nasal administration results in inhibition of gene expression in the lung. In another embodiment, the lung specific delivery of the composition is delivered in an effective amount to treat, diagnose, or prevent at least one symptom of a lung disease. Suitable lung diseases for treatment, diagnosis, or prevention include but are not limited to, lung cancers; lung inflammatory conditions such as asthma, cystic fibrosis, emphysema, bronchitis, and bronchiectasis; interstitial lung disease and interstitial fibrosis; pneumonia caused by bacterial, viral, fungal, parasitic, or mycobacteria infection; occupational lung diseases such as coal, silica, asbestos, and isocyanate related diseases; lung disease secondary to collagen vascular diseases such as systemic lupus erythematosis; rheumatoid arthritis; scleroderma; dermatomyositis; mixed connective tissue disorder; vasculitis associated lung disease such as Wegener granulomatosis and Good-pasture's Syndrome; sarcoid; and Acute Lung Injury/Acute Respiratory Distress Syndrome. In one embodiment, the nucleic acid molecule inhibits expression of RTP801 or p53.

### BRIEF DESCRIPTION OF THE DRAWINGS

FIG. 1A-C shows the results of an experiment involving the intratracheal instillation of an RTP801 expressing plasmid into mice;

FIG. 2 A-C shows the results of a short-term (7 days) cigarette smoking model in RTP801 KO and WT mice;

FIG. 3 A-C shows the results of a short-term cigarette smoking model in WT mice instilled with active anti-RTP801 (REDD14) and control (REDD8) siRNA.

FIG. 4 shows the results of experiments with RTP801 KO mice in a long-term CS model.

FIG. 5 SCID-Beige mice injected i.v. with ARE-luciferase reporter tumor cells were inhaled with Nrf2 siRNA twice during the 4th week of lung tumor growth. Control mice were inhaled with GFP siRNA. Mice were imaged before and after siRNA inhalation.

### DETAILED DESCRIPTION OF THE INVENTION

“Respiratory disorder” refers to conditions, diseases or syndromes of the respiratory system including but not limited to pulmonary disorders of all types including chronic obstructive pulmonary disease (COPD), emphysema, chronic bronchitis, asthma lung cancer, and any other lung disease or respiratory disorder disclosed herein. Emphysema and chronic bronchitis may occur in conjunction with COPD or independently.

Thus, in one embodiment, the present invention comprises a method for treatment of a subject suffering from a lung disease, which comprises administering to the subject an effective amount of a naked modified siRNA compound which inhibits expression of a gene in the form of an aerosol to the inner lung of the subject.

The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. For example, siRNA in water or PBS is naked siRNA.

Further, the siRNA is modified with one or more of the modifications disclosed herein. The siRNA compound is optionally 23 nucleotides or less in length, and may have any length specified herein as appropriate for siRNA. Further, in place of siRNA, antisense RNA or miRNA may be used. Inhibitory nucleic acid molecules which combine dsDNA, dsRNA, ssDNA or ssRNA may also be used, and said molecules may comprise any of the features disclosed herein in the context of siRNA/inhibitory RNA molecules.

The compound may be administered at a dose in the range 0.1 to 10 grams per kilogram of body weight of the subject per day; additional possible doses are disclosed herein.

The gene to be inhibited according to the present invention is preferably selected from the group consisting of RTP801, p53, TP53BP2, LRDD, CYBA, ATF3, CASP2, NOX3, HRK, C1QBP, BNIP3, MAPK8, MAPK14, Rac1, GSK3B, P2RX7, TRPM2, PARG, CD38, STEAP4, BMP2, GJA1, TYROBP, CTGF, SPP1, RTN4R, ANXA2, RHOA DUOX1, NRF2, REDD2, NOX4, MYC, PLK1, ESPL1, KEAP1, and SHC1, and particularly DUOX1 TYROBP and CTGF and particularly for the group consisting of RTP801, p53, DUOX1, TYROBP and CTGF.

The lung disease to be treated according to the present invention is a disease selected from the group consisting of COPD, lung cancer, asthma, cystic fibrosis, emphysema, bronchitis, bronchiectasis, interstitial lung disease, interstitial fibrosis, bacterial pneumonia, viral pneumonia, fungal pneumonia, parasitic pneumonia, mycobacteria-caused pneumonia, occupational lung diseases caused by agents such as coal, silica, asbestos, and isocyanates; secondary lung disease in collagen vascular diseases such as systemic lupus erythematosis, rheumatoid arthritis, scleroderma, dermatomyositis, mixed connective tissue disorder; vasculitis associated lung disease such as Wegener granulomatosis and Good-pasture's Syndrome; sarcoid; and Acute Lung Injury or Acute Respiratory Distress Syndrome.

The aerosol according to the present invention may have an average particle size of about 1-5 micrometers in diameter, optionally 1 or 2 or 3 or 4 or 5 micrometers in diameter. Further, the aerosol according to the present invention preferably has an average particle size of about 4.1-4.9 micrometers in diameter, optionally 4.4-4.7 micrometers or 4.5-4.6 micrometers in diameter. Additionally, the aerosol is preferably administered at a flow rate greater than 0.3 mL per minute. The aerosol is preferably administered orally, nasally or oro-nasally. Additional modes of administration are possible, as disclosed herein.

Suitable nucleic acids for oral, nasal or oro-nasal delivery include, but are not limited to, dsDNA, dsRNA, ssDNA, ssRNA, short interfering RNA, micro-RNA, and antisense RNA. In a preferred embodiment of the present invention, naked modified siRNA is used. Such nucleic acids can be therapeutic in that they prevent or treat one or more symptoms of a disease, or they can be diagnostic. Suitable nucleic acids for oro-nasal delivery can be constructed through a variety of methods known to one of ordinary skill in the art. The size range of the nucleic acid molecules is preferably 30 nucleotides or less in length, more preferably 23 nucleotides or less in length, although it may be possible for the molecules to be up to 60 nucleotides in length. More preferably the size range of the nucleic acid molecules is between 19 and 23 nucleotides in length (inclusive).

Any DNA that would be useful to achieve a desired effect could be used as described herein. Preferably, the DNA is double-stranded or single stranded. More preferably, the DNA is single-stranded. For example, inactivation of gene expression by nucleic acid might also be exerted by triple helix formation between genomic double-stranded DNA and an oligonucleotide. These oligonucleotides can bind with high specificity of recognition to the major groove of double helical DNA by forming Hoogsteen type bonds between the purine bases of the Watson-Crick base pairs, (i.e. between the thymidine and TA base pairs and between protonated cytosine and CG base pairs). A second motif for triple helix recognition of double-stranded DNA is comprised by a homopurine motif in which a purine-rich oligonucleotide binds to DNA antiparallel to the Watson-Crick purine strand. Pyrimidine unmodified oligodeoxynucleotides or backbone-modified oligonucleotides are able to block gene transcription in a sequence specific manner. Oligonucleotides that can bind specifically to double-helical DNA to form a local triple helix structure have been characterized for more than a decade and a wealth of information on the parameters that govern their structure and stability is available (Sun, et al. Curr. Opin. Struct. Biol. 6:327-333 (1996)). Triplex formation within promoter sites has been shown to block transcription factor access and inhibit gene activation in vitro, and several studies have demonstrated that triple helix forming oligonucleotides (TFOs) can decrease gene expression in mammalian cells in a directed way (reviewed in Seidman, et al., J Clin Invest. 112(4):487-94 (2003)). TFOs can also be used to mediate genome modification, resulting in a change in target sequence. This has the advantage of introducing permanent changes in the target sequence. It also has potential as a gene knockout tool and as a means for gene correction. Synthesis of oligonucleotides can be synthesized by any methods known to those skilled in the art.

Antisense RNA (asRNA) technology involves the down-regulation or silencing of gene expression. An “antisense” RNA molecule contains the complement of, and can therefore hybridize with, protein-encoding RNAs of the cell. Antisense oligomers have been shown to bind to messenger RNA at specific sites and inhibit the translation of the RNA into protein, splicing of mRNA or reverse transcription of viral RNA and other processing of mRNA or viral RNA.

RNA silencing is a sequence-specific RNA degradation system that is conserved in a wide range of organisms. RNA silencing is a process by which double-stranded RNA (dsRNA) silences gene expression. Two types of dsRNA involved in RNA silencing include small interfering RNAs (siRNAs) and micro-RNAs (miRNAs), short double stranded ribonucleic acids that are found in a number of organisms (see Dykxhoorn et al. 2003 Nature Reviews Mol Cell Biol 4:457-466 for a review). siRNA-like gene silencing mechanisms are functional in virtually all species, including humans. The sequences of many miRNA are known and their positions in the genome or chromosome have been published.

Although there are some differences in the expression and maturation of siRNAs and miRNAs, the final and active product is in both cases preferably a short, 19-22 nucleotide long, double-stranded RNA molecule (Dykxhoorn et al. 2003 Nature Reviews Mol Cell Biol 4:457-466 and Steinberg 2003 Scientist June 16:22-24). miRNA is synthesized from non-protein coding DNA and is metabolized from transcripts accommodating inverted repeats. The double-stranded RNA formed by foldback is processed by an RNAse III-like enzyme, highly conserved through evolution from yeast to man and higher plants, called Dicer in animals or Dicer-like in plants. These molecules can interact with the 3′-UTRs of transcripts and inhibit translation.

### Aqueous Solutions

The nucleic acid molecules can be prepared in any aqueous carrier, vehicle, or solution so as to provide a composition that is pharmaceutically suitable for in vivo administration. Methods of preparing aqueous solutions are well known to one of ordinary skill in the art. Preferably, the aqueous solution is water, or a physiologically acceptable aqueous solution containing salts and/or buffers, such as phosphate buffered saline (PBS), or any other aqueous solution acceptable for administration to a animal or human. Such solutions are well known to a person skilled in the art and include, but are not limited to, distilled water, de-ionized water, pure or ultrapure water, saline, phosphate-buffered saline (PBS), and solutions containing other buffers which are compatible with nucleic acids. The compositions may also contain sodium chloride and glucose or mannitol to make the solution isotonic. The composition may contain suitable auxiliary components such as pH, osmolarity and tonicity adjusting agents. An siRNA compound formulated in an aqueous solution is considered naked siRNA according to the present invention.

For administration via the upper respiratory tract, the composition is formulated into a solution, e.g., water or isotonic saline, buffered or unbuffered, or as a suspension, at an appropriate concentration for oro-nasal administration as an aerosol. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's Pharmaceutical Sciences 16th edition, Ed. Arthur Osol, page 1445 (1980)). One skilled in the art can readily determine a suitable saline content and pH for an innocuous aqueous solution for nasal and/or upper respiratory administration.

Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The compositions may contain minor amounts of polymers, surfactants, or other excipients well known to those of the art. In this context, “minor amounts” means no auxiliary agents or substances are present that might affect or mediate uptake of nucleic acid in the cells of the lungs.

### Administration of Compositions to the Respiratory Tract

### A. Methods of Administration

Lung-specific delivery of nucleic acid molecule formulations with little to no polymers, surfactants, or excipients as described herein, has diagnostic, prophylactic and therapeutic application for a wide range of lung diseases. Pulmonary administration can typically be completed without the need for medical intervention (self-administration), the pain often associated with injection therapy is avoided, and the amount of enzymatic and pH mediated degradation of the bioactive agent, frequently encountered with oral therapies, is significantly reduced.

The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorbtion occurs. The alveoli are covered by a thin epithelium without cilia or a mucus blanket and secrete surfactant phospholipids (J. S. Patton & R. M. Platz. Adv. Drug Del. Rev. 8:179-196 (1992)).

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung (Gonda, I. in Critical Reviews in Therapeutic Drug Carrier Systems, 6:273-313 (1990)). The inner lung, including deep lung/alveoli, is the primary target of inhaled therapeutic aerosols for systemic drug delivery.

Inhaled aerosols have been used for the treatment of local lung disorders including asthma and cystic fibrosis (Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)). Delivery of the aerosols and/or nebulized compounds may be nasal, oral, or oro-nasal, inter alia.

Aerosol dosage, formulations and delivery systems may be selected for a particular therapeutic application, as described, for example, in Gonda, I. (above); and in Moren, “Aerosol dosage forms and formulations,” in: Aerosols in Medicine, Principles, Diagnosis and Therapy, Moren, et al., Eds. Esevier, Amsterdam, 1985. The term aerosol as used herein refers to any preparation of a fine mist of particles, which can be in solution or a suspension, whether or not it is produced using a propellant. Aerosols can be produced using standard techniques, such as ultrasonication or high pressure treatment, inter alia. According to one embodiment of the present invention, the aerosol is produced using an Aeroneb nebulizer—see for example U.S. Pat. No. 6,615,824 to Power.

The formulation may be administered in an aqueous solution that is pharmaceutically acceptable for administration to the respiratory system. In preferred embodiments, the compound is administered through inhalation in a form such as liquid particles and/or solid particles. Suitable examples include, but are not limited to, an aerosol, a nebula, a mist, an atomized sample, and liquid drops. Typical apparatus which may be used for administration to humans include metered dose inhalers (MDI), nebulizers, such as Aeroneb, and instillation techniques. The formulation is administered in an amount effective to treat, prevent, or diagnose on one or more symptoms of lung disease. SiRNAs may also be administered as dry powders using a dry powder inhaler, where the particles dissolve within the lung secretions.

Various suitable devices and methods of inhalation which can be used to administer particles to a patient's respiratory tract are known in the art. Nebulizers create a fine mist from a solution or suspension, which is inhaled by the patient. The devices described in U.S. Pat. No. 5,709,202 to Lloyd, et al., or in U.S. Pat. No. 6,615,824 of Power can be used. An MDI typically includes a pressurized canister having a meter valve, wherein the canister is filled with the solution or suspension and a propellant. The solvent itself may function as the propellant, or the composition may be combined with a propellant, such as freon. The composition is a fine mist when released from the canister due to the release in pressure. The propellant and solvent may wholly or partially evaporate due to the decrease in pressure.

The compositions are preferably delivered into the lung with a pharmacokinetic profile that results in the delivery of an effective dose of the nucleic acid. As generally used herein, an “effective amount” of a nucleic acid of the invention is that amount which is able to treat one or more symptoms of a lung disease, reverse the progression of one or more symptoms of a lung disease, halt the progression of one or more symptoms of a lung disease, prevent the occurrence of one or more symptoms of a lung disease, decrease a manifestation of the disease or diagnose one or more symptoms of a lung disease in a subject to whom the compound or therapeutic agent is administered, as compared to a matched subject not receiving the compound or therapeutic agent. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (e.g. by means of an appropriate, conventional pharmacological protocol). In one embodiment, the compositions are delivered at a dose range of 0.1 to 10 grams per kilogram of body weight daily, with upper dosing limit of 0.1 kilogram per kilogram body weight daily. In a preferred embodiment the compositions are delivered at a dose range of 1 to 5 grams per kilogram of body weight daily.

One or more of these molecules can be administered to an animal (e.g., a human) to modulate expression or activity of one or more target polypeptides. A physician may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The efficacy of treatment can be monitored either by measuring the amount of the target gene mRNA (e.g. using real time PCR) or the amount of polypeptide encoded by the target gene mRNA (Western blot analysis).

### B. Patients and Diseases to be Treated

The compositions are administered to a patient in need of treatment, prophylaxis or diagnosis. The compositions can be administered to animals or humans. The lung disease may be COPD, lung cancer, a respiratory tract or lung infection, a disease of the interstitium, a disorder of gas exchange or blood circulation, a disease of the airways or a disorder of the pleura. As used herein, a “lung cancer” refers to either a primary lung tumor (for example, bronchogenic carcinoma or bronchial carcinoid) or a metastasis from a primary tumor of another organ or tissue (for example, breast, colon, prostate, kidney, thyroid, stomach, cervix, rectum, testis, bone, or melanoma). As used herein, a “respiratory tract or lung infection” refers to any bacterial, viral, fungal, or parasite infection of any part of the respiratory system. As used herein, a “disease of the interstitium” includes any disorder of the interstitium including fibrosis (for example, interstitial pulmonary fibrosis, interstitial pneumonia, interstitial lung disease, Langerhans' cell granulomatosis, sarcoidosis, or idiopathic pulmonary hemosiderosis). As used herein, a “disorder of gas exchange or blood circulation”, refers to any abnormality affecting the distribution and/or exchange of gases to/from the blood and lungs (for example, pulmonary edema, pulmonary embolism, respiratory failure (e.g., due to weak muscles), acute respiratory distress syndrome, or pulmonary hypertension). As used herein, a “disease of the airway” includes any disorder of regular breathing patterns, including disorders of genetic and environmental etiologies (for example, asthma, chronic bronchitis, bronchiolitis, cystic fibrosis, bronchiectasis, emphysema, chronic obstructive pulmonary disease, diffuse panbronchiolitis, or lymphangiomyonatosis). As used herein, a “disorder of the pleura” includes, for example, pleural effusion (e.g., hemothorax (blood into the pleural space), or emphysema (pus into the pleural space), pneumothorax (air, e.g., traumatic, spontaneous, or tension), pleurisy or pleural fibrosis or calcification.

Suitable lung diseases for treatment diagnosis, or prevention of at least one symptom of the lung disease include but are not limited to, COPD; lung cancers; lung inflammatory conditions such as asthma, cystic fibrosis, emphysema, bronchitis, and bronchiectasis; interstitial lung disease and interstitial fibrosis; pneumonia caused by bacterial, viral, fungal, parasitic, and mycobacteria infection; occupational lung diseases such as coal, silica, asbestos, and isocyanates; lung disease secondary to collagen vascular diseases such as systemic lupus erythematosis; rheumatoid arthritis; scleroderma; dermatomyositis; mixed connective tissue disorder; vasculitis associated lung disease such as Wegener granulomatosis and Good-pasture's Syndrome; sarcoid; and the syndrome of Acute Lung Injury/Acute Respiratory Distress Syndrome. In a preferred embodiment for systemic delivery, the nucleic acid inhibits expression of on or more of the following genes:p53, RTP801, Caspase 1, Caspase 2, Caspase 3, Caspase 4, Caspase 5, Caspase 6, Caspase 7, Caspase 8, Caspase 9, Caspase 10, Caspase 12, Caspase 14, Apaf-1, Nod1, Nod2, Ipaf, DEFCAP, RAIDD, RICK, Bc110, ASC, TUCAN, ARC, CLARP, FADD, DEDD, DEDD2, Cryopirin, PYC1, Pyrin, TRADD, UNC5a, UNC5b, UNC5c, ZUD, p84N5, LRDD, CDK1, CDK2, CDK4, CDK5, CDK9, PITSLRE A, CHK2, LATS1, Prk, MAP4K1, MAP4K2, STK4, SLK, GSK3alpha, GSK3beta, MEKK1, MAP3K5 (Ask1), MAP3K7, MAP3K8, MAP3K9, MAP3K10, MAP3K11, MAP3K12, DRP-1, MKK6, p38, JNK3, DAPK1, DRAK1, DRAK2, IRAK, RIP, RIP3, RIPS, PKR, IRE1, MSK1, PKCalpha, PKCbeta, PKCdelta, PKCepsilon, PKCeta, PKCmu, PKCtheta, PKCzeta, CAMK2A, HIPK2, LKB1, BTK, c-Src, FYN, Lck, ABL2, ZAP70, TrkA, TrkC, MYLK, FGFR2, EphA2, AATYK, c-Met, RET, PRKAA2, PLA2G2A, SMPD1, SMPD2, SPP1, FAN, PLCG2, 1P6K2, PTEN, SHIP, AIF, AMID, Cytochrome c, Smac, HtrA2, TSAP6, DAP-1, FEM-1, DAP-3, Granzyme B, DIO-1, DAXX, CAD, CIDE-A, CIDE-B, Fsp27, Apel, ERCC2, ERCC3, BAP31, Bitl, AES, Huntingtin, HIP1, hSir2, PHAP1, GADD45b, GADD34, RAD21, MSH6, ADAR, MBD4, WW45, ATM, mTOR, T1P49, diubiquitin/FAT10, FAF1, p193, Scythe/BAT3, Amida, IGFBP-3, TDAG51, MCG10, PACT, p52/RAP, ALG2, ALG3, presenelin-1, PSAP, AlP1/Alix, ES18, mda-7, p14ARF, ANT1, p331NG1, p331NG2, p53AIP1, p53DINP1, MGC35083, NRAGE, GRIM19, lipocalin 2, glycodelin A, NADE, Porimin, STAG1, DAB2, Galectin-7, Galectin-9, SPRC, FLJ21908, WWOX, XK, DKK-1, Fzd1, Fzd2, SARP2, axin 1, RGS3, DVL1, NFkB2, 1 kBalpha, NF-ATC1, NF-ATC2, NF-ATC4, zf3/ZNF319, Egr1, Egr2, Egr3, Spl, TIEG, WT1, Zac1, Icaros, ZNF148, ZK1/ZNF443, ZNF274, WIG1, HIVEP1, HIVEP3, Fliz1, ZPR9, GATA3, TR3, PPARG, CSMF, RXRa, RARa, RARb, RARg, T3Ra, ERbeta, VDR, GR/GCCR, p53, p73alpha, p63(human Eta alpha, ta beta, ta gamma, da alpha, da beta, da gamma1, 53BP2, ASPP1, E2F1, E2F2, E2F3, HIF1 alpha, TCF4, c-Myc, Max, Mad, MITF, Id2, Id3, Id4, c-Jun, c-Fos, ATF3, NF-1L6, CHOP, NRF1, c-Maf, Bach2, Msx2, Csx, Hoxa5, Ets-1, PU1/Spi1, Ets-2, ELK1, TEL1, c-Myb, TBX5, IRF1, IRF3, IRF4, IRF9, AP-2 alpha, FKHR, FOXO1A, FKHRL1, FOXO3a, AFX1, MLLT7, Tip60, BTG1, AUF1, HNRPD, TIA1, NDG1, PCBP4, MCG10, FXR2, TNFR2, LTbR, CD40, CD27, CD30, 4-1BB, TNFRSF19, XEDAR, Fn14, OPG, DcR3, FAS, TNFR1, WSL-1, p75NTR, DR4, DR5, DR6, EDAR, TNF alpha, FAS ligand, TRAIL, Lymphotoxin alpha, Lymphotoxin beta, 4-1BBL, RANKL, TL1, TWEAK, LIGHT, APRIL, IL-1-alpha, IL-1-beta, IL-18, FGF8, IL-2, IL-21, IL-5, IL-4, IL-6, LIF, IL-12, IL-7, IL-10, IL-19, IL-24, IFN alpha, IFN beta, IFN gamma, M-CSF, prolactin, TLR2, TLR3, TLR4, MyD88, TRIF, RIG-1, CD14, TCR alpha, CD3 gamma, CD8, CD4, CD7, 0)19, CD28, CTLA4, SEMA3A, SEMA3B, HLA-A, HLA-B, HLA-L, HLA-DMalpha, CD22, CD33, CALL, DCC, ICAM1, ICAM3, CD66a, PVR, CD47, CD2, Thy-1, SIRPa1, CD5, E-cadherin, ITGAM, ITGAV, CD18, ITGB3, CD9, IgE Fc R beta, CD82, CD81, PERP, CD24, CD69, KLRD1, galectin 1, B4GALT1, C1q alpha, C5R1, MIP1alpha, MIP1beta, RANTES, SDF1, XCL1, CCCKR5, OIAS/OAS1, INDO, MxA, IFI16, AIM2, iNOS, HB-EGF, HGF, MIF, TRAF3, TRAF4, TRAF6, PAR-4, IKKGamma, FIP2, TXBP151, FLASH, TRF1, IEX-1S, Dok1, BLNK, CIN85, Bif-1, HEF1, Vav1, RasGRP1, POSH, Rac1, RhoA, RhoB, RhoC, ALG4, SPP1, TRIP, SIVA, TRABID, TSC-22, BRCA1, BARD1, 53BP1, MDC1, Mdm4, Siah-1, Siah-2, RoRet, TRIM35, PML, RFWD1, DIP1, Socs1, PARC, USP7, CYLD, SOX9, ASPP1, CTSD, CAPNS1, FAS, FAS ligand, HES1, HESS, ID1, ID2, ID3 CDKN1B, CDKN2A; and particularly of RTP801, p53, TP53BP2, LRDD, CYBA, ATF3, CASP2, NOX3, HRK, C1QBP, BNIP3, MAPK8, MAPK14, Rac1, GSK3B, P2RX7, TRPM2, PARG, CD38, STEAP4, BMP2, GJA1, TYROBP, CTGF, SPP1, RTN4R, ANXA2, RHOA DUOX1, NRF2, REDD2, NOX4, MYC, PLK1, ESPL1, KEAP1, and SHC1; and even more particularly of RTP801, p53, DUOX1 TYROBP and CTGF.

C. siRNAs to be Administered

Any double-stranded oligoribonucleotides (e.g. siRNAs) which down-regulates a mammalian gene can be administered according to the methods of the present invention An siRNA to be administered according to the present invention is a duplex oligoribonucleotide in which the sense strand is derived from the mRNA sequence of a mammalian gene, optionally the genes disclosed herein, and the antisense strand is complementary to the sense strand. In general, some deviation from the target mRNA sequence is tolerated without compromising the siRNA activity (see e.g. Czauderna et al., 2003, NAR 31(11), 2705-2716). An siRNA of the invention inhibits gene expression on a post-transcriptional level with or without destroying the mRNA. Without being bound by theory, siRNA may target the mRNA for specific cleavage and degradation and/or may inhibit translation from the targeted message.

As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide.

In some embodiments the oligoribonucleotide according to the present invention comprises modified siRNA. In various embodiments the siRNA comprises an RNA duplex comprising a first strand and a second strand, whereby the first strand comprises a ribonucleotide sequence at least partially complementary to about 18 to about 40 consecutive nucleotides of a target nucleic acid, and the second strand comprises ribonucleotide sequence at least partially complementary to the first strand and wherein said first strand and/or said second strand comprises a plurality of groups of modified ribonucleotides having a modification at the 2′-position of the sugar moiety whereby within each strand each group of modified ribonucleotides is flanked on one or both sides by a group of flanking ribonucleotides whereby each ribonucleotide forming the group of flanking ribonucleotides is selected from an unmodified ribonucleotide or a ribonucleotide having a modification different from the modification of the groups of modified ribonucleotides.

In one embodiment, the group of modified ribonucleotides and/or the group of flanking ribonucleotides comprise a number of ribonucleotides selected from the group consisting of an integer from 1 to 12. Accordingly, the group thus comprises one nucleotide, two nucleotides, three nucleotides, four nucleotides, five nucleotides, six nucleotides, seven nucleotides, eight to nucleotides, nine nucleotides, ten nucleotides, eleven nucleotides or twelve nucleotides.

The groups of modified nucleotides and flanking nucleotides may be organized in a pattern on at least one of the strands. In some embodiments the first and second strands comprise a pattern of modified nucleotides. In another embodiment, only one strand comprises a pattern of modified nucleotides. In various embodiments the pattern of modified nucleotides of said first strand is identical relative to the pattern of modified nucleotides of the second strand.

In other embodiments the pattern of modified nucleotides of said first strand is shifted by one or more nucleotides relative to the pattern of modified nucleotides of the second strand.

In some preferred embodiments the middle ribonucleotide in the antisense strand is an unmodified nucleotide. For example, in a 19-oligomer antisense strand, ribonucleotide number 10 is unmodified; in a 21-oligomer antisense strand, ribonucleotide number 11 is unmodified; and in a 23-oligomer antisense strand, ribonucleotide number 12 is unmodified. The modifications or pattern of modification, if any, of the siRNA must be planned to allow for this. The modifications on the 2′ moiety of the sugar residue include amino, fluoro, alkoxy e.g. methoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O—, S—, or N-alkyl; O-, S, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP 0 586 520 B1 or EP 0 618 925 B1.

In some embodiments the siRNA is blunt ended, at one or both ends. More specifically, the siRNA may be blunt ended on the end defined by the 5′-terminus of the first strand and the 3′-terminus of the second strand, or the end defined by the 3′-terminus of the first strand and the 5′-terminus of the second strand. In other embodiments at least one of the two strands may have an overhang of at least one nucleotide at the 5′-terminus. At least one of the strands may also optionally have an overhang of at least one nucleotide at the 3′-terminus. The overhang may consist of from about 1 to about 5 consecutive nucleotides. A nucleotide of the overhang may be a modified or unmodified ribonucleotide or deoxyribonucleotide.

The length of RNA duplex is from about 18 to about 40 ribonucleotides, preferably 19, 21 or 23 ribonucleotides. Further, the length of each strand may independently have a length selected from the group consisting of about 15 to about 40 bases, preferably 18 to 23 bases and more preferably 19, 21 or 23 ribonucleotides.

Additionally, the complementarity between said first strand and the target nucleic acid may be perfect. In some embodiments, the strands are substantially complementary, i.e. having one, two or up to three mismatches between said first strand and the target nucleic acid. Substantially complementary refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence.

In certain embodiments the first strand and the second strand each comprise at least one group of modified ribonucleotides and at least one group of flanking ribonucleotides, whereby each group of modified ribonucleotides comprises at least one ribonucleotide and whereby each group of flanking ribonucleotides comprises at least one ribonucleotide, wherein each group of modified ribonucleotides of the first strand is aligned with a group of flanking ribonucleotides on the second strand, and wherein the 5′ most terminal ribonucleotide is selected from a group of modified ribonucleotides, and the 3′ most terminal ribonucleotide of the second strand is a selected from the group of flanking ribonucleotide. In some embodiments each group of modified ribonucleotides consists of a single ribonucleotide and each group of flanking ribonucleotides consists of a single nucleotide.

In yet other embodiments the ribonucleotide forming the group of flanking ribonucleotides on the first strand is an unmodified ribonucleotide arranged in a 3′ direction relative to the ribonucleotide forming the group of modified ribonucleotides, and the ribonucleotide forming the group of modified ribonucleotides on the second strand is a modified ribonucleotide which is arranged in 5′ direction relative to the ribonucleotide forming the group of flanking ribonucleotides. In some embodiments the first strand of the siRNA comprises five to about twenty, eight to twelve, preferably nine to twelve, groups of modified ribonucleotides, and the second strand comprises seven to eleven, preferably eight to eleven, groups of modified ribonucleotides.

The first strand and the second strand may be linked by a loop structure, which may be comprised of a non-nucleic acid polymer such as, inter alfa, polyethylene glycol. Alternatively, the loop structure may be comprised of a nucleic acid, including modified and non-modified ribonucleotides and modified and non-modified deoxyribonucleotides.

Further, the 5′-terminus of the first strand of the siRNA may be linked to the 3′-terminus of the second strand, or the 3′-terminus of the first strand may be linked to the 5′-terminus of the second strand, said linkage being via a nucleic acid linker or a non-nucleic acid linker. In certain embodiments a nucleic acid linker has a length of between about 2-100 nucleic acids, preferably about 2 to about 30 nucleic acids.

In various embodiments, the present invention provides for administration of a compound having the structure:

| | |
|---|---|
| 5′ (N)x-Z 3′ | (antisense strand) |
| 3′Z′-(N′)y 5′ | (sense strand) |

wherein each N and N′ is a ribonucleotide which may be modified or unmodified in its sugar residue; and each of (N)ₓ and (N′)_{y} is an oligomer in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present;
and wherein the sequence of (N′)_{y} comprises a sense sequence having substantial identity to about 18 to about 40 consecutive ribonucleotides in a mammalian mRNA. In preferred embodiments the mRNA is selected from the mRNA transcribed from any one of the genes disclosed herein.

In some embodiments the compound comprises a phosphodiester bond.

In various embodiments the compound comprises ribonucleotides wherein x=y and wherein x is an integer selected from the group consisting of 18, 19, 20, 21, 22 and 23. In certain embodiments x=y=19 or x=y=23.

In some embodiments the compound is blunt ended, for example wherein Z and Z′ are both absent. In an alternative embodiment, the compound comprises at least one 3′ overhang, wherein at least one of Z or Z′ is present. Z and Z′ can be independently comprise one or more covalently linked modified or non-modified nucleotides, as described infra, for example inverted dT or dA; dT, LNA (locked nucleic acids), mirror nucleotide and the like. In some embodiments each of Z and Z′ are independently selected from dT and dTdT.

In some embodiments the compound comprises one or more ribonucleotides unmodified in their sugar residues. In other embodiments the compound comprises at least one ribonucleotide modified in the sugar residue. In some embodiments the compound comprises a modification at the 2′ position of the sugar residue. Modifications in the 2′ position of the sugar residue include amino, fluoro, alkoxy and alkyl moieties. In certain preferred embodiments the alkoxy modification is a methoxy moiety at the 2′ position of the sugar residue (2′-O-methyl; 2′-O-Me; 2′-O—CH₃).

In some embodiments the compound comprises modified alternating ribonucleotides in one or both of the antisense and the sense strands. In certain embodiments the compound comprises modified alternating ribonucleotides in the antisense and the sense strands. In some preferred embodiments the middle ribonucleotide of the antisense strand is not modified; e.g. ribonucleotide in position 10 in a 19-mer strand.

In certain embodiments the present invention provides for administration of a compound having the structure

| | |
|---|---|
| 5′(N)x 3′ | antisense strand |
| 3′(N′)y 5′ | sense strand |

wherein each of x and y=19 and (N)ₓ and (N′)_{y} are fully complementary;
wherein alternating ribonucleotides in (N)ₓ and (N′)_{y} are modified to result in a 2′-O-methyl modification in the sugar residue of the ribonucleotides;
wherein each N at the 5′ and 3′ termini of (N)ₓ are modified;
wherein each N′ at the 5′ and 3′ termini of (N′)_{y} are unmodified;
wherein each of (N)ₓ and (N′)_{y} is present in or complementary to an mRNA transcribed from a mammalian gene, optionally one of the genes disclosed herein.

(N)ₓ and (N′)_{y} may be phosphorylated or non-phosphorylated at the 3′ and 5′ termini.

In certain embodiments of the invention, alternating ribonucleotides are modified in the 2′ position of the sugar residue in both the antisense and the sense strands of the compound. In particular the exemplified siRNA has been modified such that a 2′-O-methyl (Me) group was present on the first, third, fifth, seventh, ninth, eleventh, thirteenth, fifteenth, seventeenth and nineteenth nucleotide of the antisense strand, whereby the very same modification, i.e. a 2′-β-Me group, was present at the second, fourth, sixth, eighth, tenth, twelfth, fourteenth, sixteenth and eighteenth nucleotide of the sense strand. Additionally, it is to be noted that these particular siRNA compounds are also blunt ended.

In certain embodiments the alternating compounds being administered having ribonucleotides modified in one or both of the antisense and the sense strands of the compound, for 19-mers and 23-mers the ribonucleotides at the 5′ and 3′ termini of the antisense strand are modified in their sugar residues, and the ribonucleotides at the 5′ and 3′ termini of the sense strand are unmodified in their sugar residues. For 21-mers the ribonucleotides at the 5′ and 3′ termini of the sense strand are modified in their sugar residues, and the ribonucleotides at the 5′ and 3′ termini of the antisense strand are unmodified in their sugar residues. As mentioned above, it is preferred that the middle nucleotide of the antisense strand is unmodified.

According to one preferred embodiment of the invention, the antisense and the sense strands of the siRNA being administered are phosphorylated only at the 3′-terminus and not at the 5′-terminus. According to another preferred embodiment of the invention, the antisense and the sense strands are non-phosphorylated. According to yet another preferred embodiment of the invention, the 5′ most ribonucleotide in the sense strand is modified to abolish any possibility of in vivo 5′-phosphorylation.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol. 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

The features of the present invention disclosed in the specification, the claims and/or the drawings may both separately and in any combination thereof be material for realizing the invention in various forms thereof.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Nothing herein is to be construed as an admission that the invention is not entitled to antedate such disclosure by virtue of prior invention.

### Example 1

### Models and Results Relating to COPD and Emphysema

As indicated below, an exemplary siRNA (termed REDD14) in aqueous solution directed against gene RTP801 (see co-assigned patent publication No WO06/023544A2 and co-assigned application No. PCT/US2007/01468 which are hereby incorporated by reference in their entirety) was tested in the following animal models:
Cigarette smoke-induced emphysema model: chronic exposure to cigarette smoke causes emphysema in several animals such as, inter alia, mouse, guinea pig.
Lung protease activity as a trigger of emphysema.
VEGFR inhibition model of emphysema.
Bronchial instillation with human neutrophil/pancreatic elastase in rodents.
MMP (matrix metalloprotease)-induced enphysema.
Inflammation-induced emphysema.

Additionally, emphysema models may be generated through genetic means (e.g., mice carrying the TSK mutation), and emphysematous animals may be generated by known modifiers of susceptibility to emphysema such as, inter alia, lung injury, alveolar hypoplasia, hyperoxia, glucocorticoid treatment and nutrition.

### A. Evaluation of the Influence of Lack of RTP801 on Disease Development in Mouse Models of Emphysema (Using RTP801 Knockout Mice)

(1) Cigarette smoking (CS) induced inflammation and apoptosis was initiated in 5 RTP801 KO and 5 control wild type 4 months old male mice. The mice were subjected to intense CS (as described in Rangasamy et al., see above) for 7 days. KO and WT non-treated mice from the VEGFR inhibition experiment above also served as non-treated control groups for this experiment. The lungs were subsequently agarose-inflated, fixed and imbedded in paraffin, and development oxidative stress in the KO mice was assessed by:
   a) immunohistochemical localization and quantitation of 8-oxo-dG in the lung sections;
   b) immunohistochemical localization and quantitation of active caspase 3 in the lung sections using specific antibodies, or quantitative evaluation of the number of TUNEL-positive cells;
   c) measurement of ceramide concentration in the lung extracts;
   d) measurement of caspase activity in the lung extracts.
(2) Long-term cigarette smoking in the KO mice.

6 KO and 6 age-matched WT female mice were subjected to intense cigarette smoking (5 hrs a day) during a period of 6 months. The mice were then sacrificed, and average interseptal diameter (a parameter of emphysema development) was evaluated using a morphometric approach.

B. Evaluation of the Influence of Lack of RTP801 on Disease Progression in Mouse Models of Emphysema by Inhibiting Endogenous RTP801 Employing Intralung Delivery of RTP801-Inactivating siRNA

CS-induced inflammation was induced by 7 day smoking in 2 groups of C57BL6 mice, 10 mice per group. Group 1: CS+delivery of control siRNA (REDD8) siRNA; Group 2: CS+RTP801 siRNA (REDD14). Control groups of mice were instilled with either type of siRNA but kept in room air conditions. The animals were evaluated as in the above experiment with the Knock-Out mice.

### Methods

### Exposure to Cigarette Smoking (CS)

Exposure was carried out (7 h/day, 7 days/week) by burning 2R4F reference cigarettes (2.45 mg nicotine per cigarette; purchased from the Tobacco Research Institute, University of Kentucky, Lexington, Ky., USA) using a smoking machine (Model TE-10, Teague Enterprises, Davis, Calif., USA). Each smoldering cigarette was puffed for 2 s, once every minute for a total of eight puffs, at a flow rate of 1.05 L/min, to provide a standard puff of 35 cm³. The smoke machine was adjusted to produce a mixture of sidestream smoke (89%) and mainstream smoke (11%) by burning five cigarettes at one time. Chamber atmosphere was monitored for total suspended particulates and carbon monoxide, with concentrations of 90 mg/m3 and 350 ppm, respectively.

### Morphologic and Morphometric Analyses

After exposing the mice to CS or instillation of RTP801 expressing plasmid, the mice were anesthetized with halothane and the lungs were inflated with 0.5% low-melting agarose at a constant pressure of 25 cm as previously described⁶. The inflated lungs were fixed in 10% buffered formalin and embedded in paraffin. Sections (5 μm) were stained with hematoxylin and eosin. Mean alveolar diameter, alveolar length, and mean linear intercepts were determined by computer-assisted morphometry with the Image Pro Plus software (Media Cybernetics, Silver Spring, Md., USA). The lung sections in each group were coded and representative images (15 per lung section) were acquired by an investigator masked to the identity of the slides, with a Nikon E800 microscope, 20× lens.

### Bronchoalveolar Lavage (BAL) and Phenotyping

Following exposure to CS or instillation of RTP801 expressing plasmid, the mice were anesthetized with sodium pentobarbital. The BAL fluid collected from the lungs of the mice was centrifuged (500 g at 4° C.), and the cell pellet was resuspended in phosphate-buffered saline. The total number of cells in the lavage fluid was determined, and 2×10⁴ cells were cytocentrifuged (Shandon Southern Products, Pittsburgh, Pa., USA) onto glass slides and stained with Wright-Giemsa stain. Differential cell counts were performed on 300 cells, according to standard cytologic techniques.

### Identification of Alveolar Apoptotic Cell Populations in the Lungs.

To identify the different alveolar cell types undergoing apoptosis in the lungs, an immunohistochemical staining of active caspase 3 was performed in the lung sections from the room air (RA) as well as CS exposed mice. To identify the apoptotic type II epithelial cells in the lungs, after active caspase 3 labeling, the lung sections were incubated first with anti-mouse surfactant protein C (SpC) antibody and then with an anti-rabbit Texas red antibody. Apoptotic endothelial cells were identified by incubating the sections first with the anti-mouse CD 31 antibody and then with the biotinylated rabbit anti-mouse secondary antibody. The lung sections were rinsed in PBS and then incubated with the streptavidin-Texas red conjugated complex. The apoptotic macrophages in the lungs were identified by incubating the sections first with the rat anti-mouse Mac-3 antibody and then with the anti-rat Texas red antibody. Finally, DAPI was applied to all lung sections, incubated for 5 minutes, washed and mounted with Vectashield HardSet mounting medium. DAPI and fluorescein were visualized at 330-380 nm and 465-495 nm, respectively. Images of the lung sections were acquired with the Nikon E800 microscope, 40× lens.

### Immunohistochemical Localization of Active Caspase-3

Immunohistochemical staining of active caspase-3 assay was performed using anti-active caspase-3 antibody and the active caspase-3-positive cells were counted with a macro, using Image Pro Plus program. The counts were normalized by the sum of the alveolar profiles herein named as alveolar length and expressed in μm. Alveolar length correlates inversely with mean linear intercept, i.e., as the alveolar septa are destroyed, mean linear intercepts increases as total alveolar length, i.e., total alveolar septal length decreases.

### Caspase 3 Activity Assay

The activity of caspase-3 activity was measured in lung tissue extracts using a fluorometric assay according to the manufacturer's instructions. Snap-frozen lung tissue (n=3 per group) was homogenized with the assay buffer, followed by sonication and centrifugation at 800×g. After removal of nuclei and cellular debris, the supernatant (300 μg protein) was then incubated with the pro-fluorescent substrate at room temperature for 1 h and the fluorescence intensity was measured utilizing a Typhoon phosphoimager (Amersham Biosciences, Inc., Piscataway, N.J., USA). The results are expressed as the rate of specific caspase-3 substrate cleavage, expressed in units of caspase 3 enzymatic activity, normalized by total protein concentration. Active recombinant caspase 3 was utilized as the assay standard (0-4 U). Tissue lysates without substrate, assay buffer alone, and lysates with caspase 3 inhibitor were utilized as negative controls.

Immunohistochemical Localization of 8-oxo-dG

For the immunohistochemical localization and quantification of 8-oxo-dG, lung sections from the mice exposed to CS or instilled with RTP801 expressing plasmid were incubated with anti-8-oxo-dG antibody and stained using InnoGenex™ Iso-1HC DAB kit using mouse antibodies. The 8-oxo-dG-positive cells were counted with a macro (using Image Pro Plus), and the counts were normalized by alveolar length as described.

Instillation of Plasmid DNA into Mouse Lungs

Plasmid DNA of RTP801 expressing and control vectors were prepared under endotoxin-free DNA isolation kit. For intra-tracheal instillation, 50 ug of plasmid DNA was delivered in 80 ul sterile perfluorocarbon. The oxygen carrying properties of perfluorocarbon make it well-tolerated at these volumes, while its physical-chemical properties allow for extremely efficient distal lung delivery when instilled intratracheally. Mice were anesthetized by brief inhalational halothane exposure, the tongue was gently pulled forward by forceps and the trachea instilled with perfluorocarbon solution applied at the base of the tongue via a blunt angiocatheter.

Instillation of siRNA into Mouse Lungs.

Mice were anesthetized with an intra-peritoneal injection of Ketamine/Xylazine (115/22 mg/kg). 50 μg of siRNA was instilled intranasally in 50 μl volume of 0.9% NaCl by delivering five consecutive 10 μl portions. At the end of the intranasal instillation, the mouse's head was held straight up for 1 minute to ensure that all the instilled solution drained inside.

For further information, see: Rangasamy T, Cho C Y, Thimmulappa, R K, Then L, Srisuma S S, Kensler T W, Yamamoto M, Petrache I, Tuder R M, Biswal S. Submitted to Journal of Clinincal Investigation; Yasunori Kasahara, Rubin M. Tuder, Carlyne D. Cool, David A. Lynch, Sonia C. Flores, and Norbert F. Voelkel. Am J Respir Crit. Care Med Vol 163. pp 737-744, 2001; Yasunori Kasahara, Rubin M. Tuder, Laimute Taraseviciene-Stewart, Timothy D. Le Cras, Steven Abman, Peter K. Hirth, Johannes Waltenberger, and Norbert F. Voelkel. J. Clin. Invest. 106:1311-1319 (2000); and a review on the topic: Robin M. Tuder, Sharon McGrath and Enid Neptune, The pathological mechanisms of emphysema models: what do they have in common?, Pulmonary Pharmacology & Therpaeutics 2002.

### Results

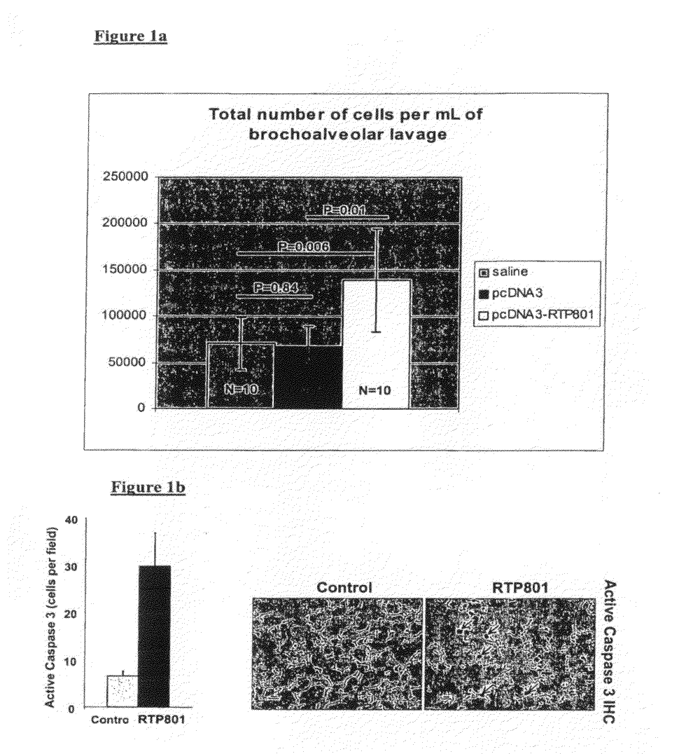

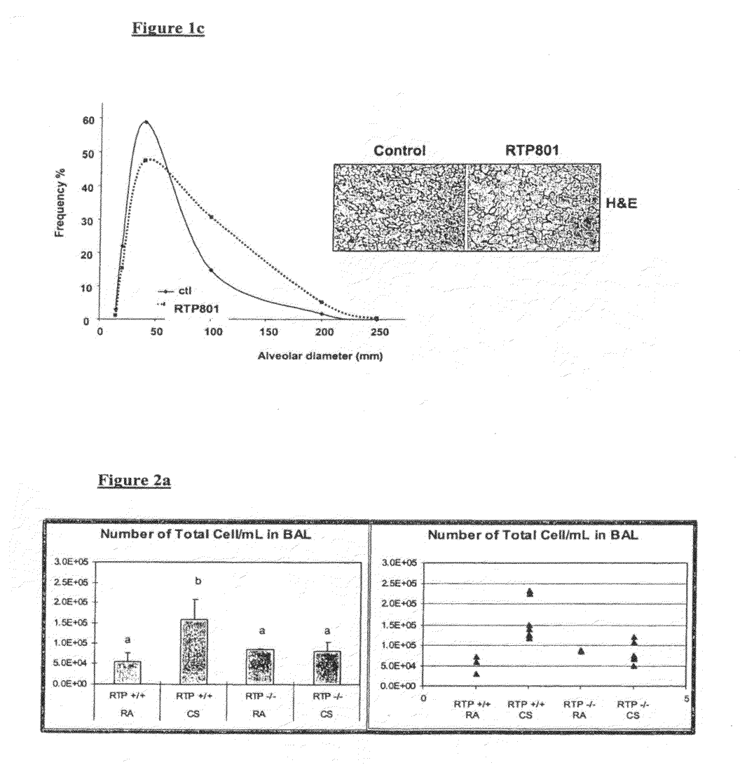

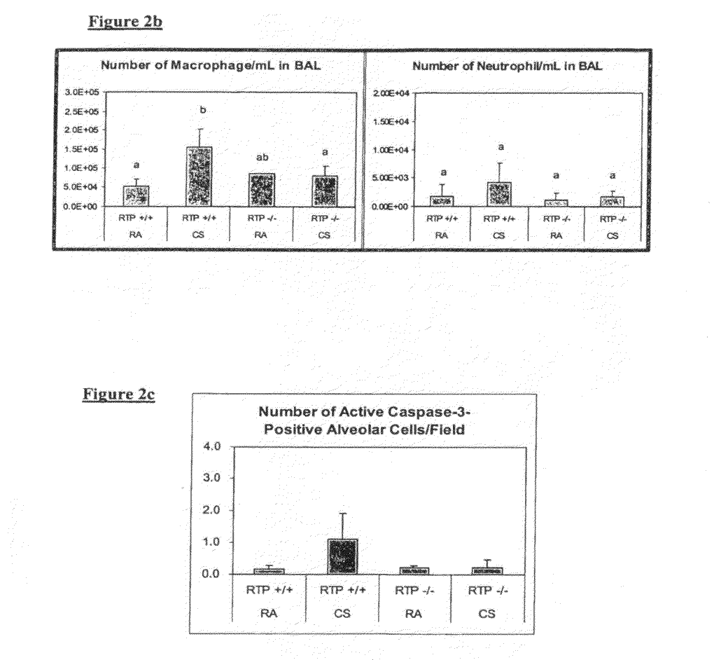

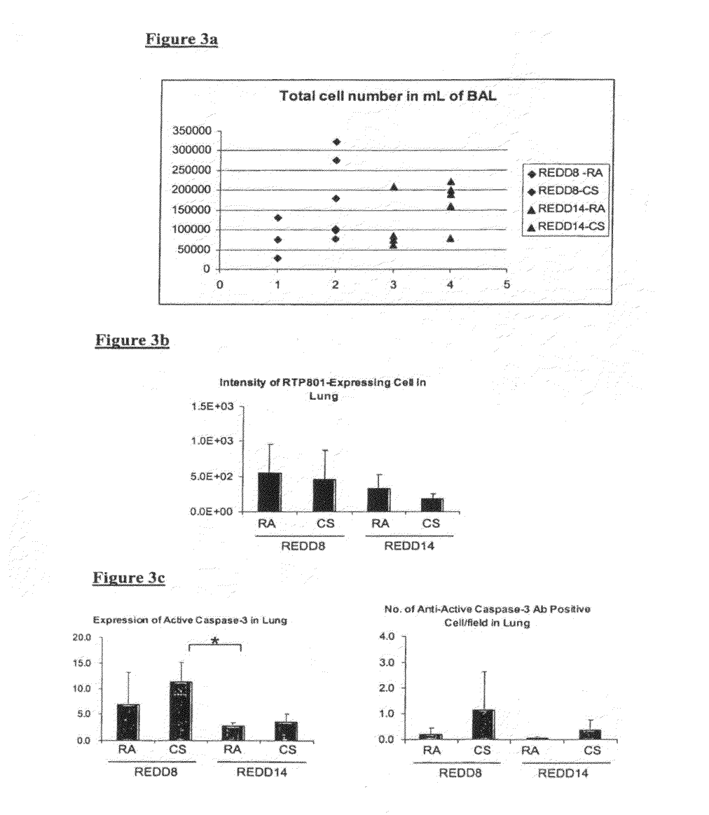

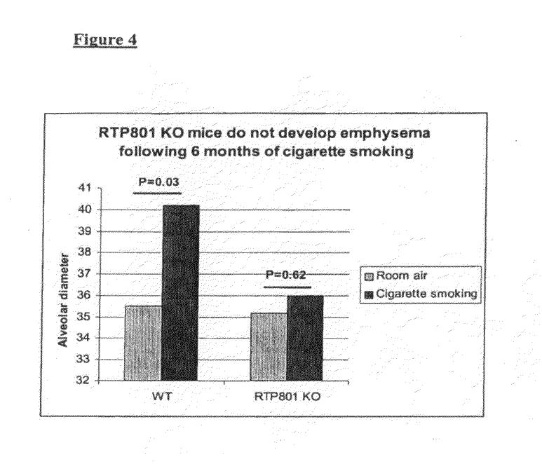

1. Instillation of an RTP801 expressing plasmid results in an emphysema-like phenotype in mouse lungs which is evident by (1) inctease in bronchoalveolar lavage cell counts (FIG. 1a); (2) apoptosis of lung septal cells (FIG. 1b) and increase in the alveolar diameter (FIG. 1c).
2. Instillation of RTP801 siRNA (REDD14) resulted in reduction of RTP801 expression in the lungs (FIG. 3b).
3. RTP801 KO mice were protected from emphysema development following 6 months of cigarette smoking as evident by the lack of enlargement of alveolar diameter. (FIG. 4).
4. RTP801 KO mice were protected from cigarette smoking induced inflammation as evident by reduced number of inflammatory bronchoalveolage cells following 1 week of cigarette smoking (FIG. 2, a-b).
5. RTP801 KO mice were protected from cigarette smoking induced apoptosis of lung septal cells as evidenced by lung section staining for activated caspase (FIG. 2c).
6. REDD14-instilled mice were partially protected from cigarette smoking induced inflammation as evident by reduced number of inflammatory bronchoalveolage cells following 1 week of cigarette smoking (FIG. 3a).
7. REDD14-instilled mice were partially protected from cigarette smoking induced apoptosis of lung septal cells as evidenced by lung section staining for activated caspase and by immunoblotting of lung extracts with anti-activated caspase 3 antibodies ((FIG. 3c) Thus siRNA was successfully delivered to the lung and prevented disease progression in various mouse models of emphysema

### Example 2

### Pharmacokinetics and Tissue Distribution of siRNA (REDD14Cy3.5 and I5NP) in Cynomolgus Monkeys Following Single Oronasal Inhalation Administration of a Nebulised Aerosol Formulation

### Summary

Male cynomolgus monkeys were administered single oronasal inhalation doses of a nebulised aerosol formulation of fluorescent-labelled REDD14Cy3.5 and/or I5NP siRNAs. Following dosing, the concentration of I5NP in plasma, the non-compartmental disposition kinetics of I5NP in plasma, the qualitative distribution of REDD14Cy3.5 in lungs and the semi-quantitative distribution of I5NP in lungs were determined.

Sequence of siRNAs used in the study:

| | |
|---|---|
| REDD14Cy3.5 | |
| Sense | GUGCCAACCUGAUGCAGCU |
| Antisense | AGCUGCAUCAGGUUGGCAC |
| 15NP | |
| Sense | GAGAAUAUUUCACCCUUCA |
| Antisense | UGAAGGGUGAAAUAUUCUC |

REDD14 is an exemplary RTP801 inhibiting siRNA; I5NP is an exemplary p53 inhibiting siRNA.

Three separate dose treatments were administered to the animals as detailed below. The theoretical inhalation achieved doses of siRNA were calculated from the respiratory minute volume (measured during pre-treatment), the aerosol concentration of the test article (measured gravimetrically during pre-treatment calibration of the nebuliser), the treatment time, the total aerosol depostion fraction (assumed as 100%) and the animal bodyweights on the day before each dose administration.

| | | | Gravimetric | Exposure | | Body | Theoretical |
|---|---|---|---|---|---|---|---|
| Treatment | Animal | RMV | concentration | duration | | weight | achieved dose |
| number | number | (L/min) | (mg/L)^{a} | (min) | Deposition | (kg) | (mg/kg/session) |
| 1 | 101 | 2.70 | 2.036 | 15 | 1 | 2.80 | 29.45 |
| | 102 | 2.66 | | 15 | | 2.60 | 31.24 |
| | 103 | 4.16 | | 10 | | 2.70 | 31.37 |
| | 106 | 5.58 | | 10 | | 3.60 | 31.56 |
| 2 | 101 | 2.70 | 2.036 | 15 | 1 | 2.80 | 29.45 |
| | 102 | 2.66 | | 13^{b} | | 2.80 | 25.14 |
| 3 | 103 | 4.16 | 2.036 | 10 | 1 | 2.90 | 29.21 |
| | 104 | 3.72 | | 10 | | 2.30 | 32.93 |
| | 105 | 4.53 | | 12 | | 3.80 | 29.13 |

^{a}Based on Pretreatment Results
^{b}Animal 102 received 13 minutes of a scheduled 15 minute exposure

No treatment-related clinical signs were observed in any of the monkeys following inhalation doses of I5NP or a mixture of I5NP and REDD14Cy3.5 siRNAs.

Following Treatment 1 (dosing with I5NP only), blood samples were collected at various times post-dose and I5NP concentrations were measured in plasma using a hybridisation assay. The highest mean concentrations of I5NP in plasma (21.0±5.01 ng/mL) were observed at the first time point analysed post-dose (15 minutes post-end of dosing), indicating that absorption of I5NP into the systemic circulation from the inhaled dose was rapid. Concentrations in plasma were low and declined rapidly up to 8 h or 24 h post-end of dosing, after which they were below the limit of quantitation (0.10 ng/mL).

Non-compartmental pharmacokinetic parameters were calculated for I5NP concentrations in monkey plasma and these are presented below:

| Pharmacokinetic | | Animal Number | | | |
|---|---|---|---|---|---|
| Parameter | Units | 101 | 102 | 103 | 106 |
| tₘₐₓ | h | 0.5 | 0.5 | 0.42 | 0.42 |
| Cₘₐₓ | ng/mL | 26.3 | 18.0 | 24.0 | 15.6 |
| tₗₐₛₜ | h | 24.25 | 8.25 | 8.17 | 8.17 |
| AUC₀₋ₜₗₐₛₜ | ng · h/mL | 70.3 | 27.1 | 42.4 | 15.8 |
| kₑₗ | h⁻¹ | 0.121 | 0.467 | 0.252 | 0.277 |
| t_{1/2} | h | 5.74 | 1.48 | 2.75 | 2.50 |
| AUC_{0-inf} | ng · h/mL | 72.0 | 27.3 | 46.8 | 16.1 |
| Extrapolation | % | 2.36 | 0.824 | 9.40 | 2.26 |

There was a considerable degree of variation between individual animals, as demonstrated by the variation in the half-life of elimination (i.e. from 1.5 to 5.7 h) and the AUC_{0-inf} (range 16.1 to 72.0 ng.h/mL).

Treatment 2 (a mixture of I5NP and fluorescent labeled REDD14Cy3.5 siRNAs, in a ratio of 9:1) was administered to animals 101 and 102 following a wash-out period of approximately 2 weeks. Animals were euthanized and lungs collected at approximately 30 minutes post-end of dosing. The lungs and right kidney from animal 102 were collected intact, fixed in 4% (v/v) paraformaldehyde and sent for fluorescence analysis. Fluorescent material was detected in all sections taken from all lobes of the lungs. Fluorescent label was observed radiating from the bronchioles outward into the alveolar walls. The strongest signals appeared to be associated with the right caudal lobe and the caudal section of the left cranial lobe. There was little label detected in the right kidney with, at most, a slight residual staining being observed in the lumen of the kidney tubules.

The lungs from animal 101 were removed and each lobe of the lungs was dissected to provide a sample of bronchiolar tissue and the remaining peripheral lung tissue. These samples were stored frozen (approximately −80° C.) until required for analysis of I5NP concentrations by hybridization analysis. The results were considered to be semi-quantitative since a specific assay for monkey lung was not developed. At approximately 30 minutes post-end of dosing, I5NP was found in high concentrations in both bronchioles and in peripheral tissues of all lobes of each lung. Concentrations were similar throughout all the lobes and were generally higher in the peripheral tissues than in the bronchiolar samples.

Treatment 3, comprising I5NP only, was administered to animals 103, 104 and 105 approximately 3 weeks following Treatment 2. Blood samples were collected at 15 minutes post-end of dosing from all animals and at the terminal time point for each individual monkey. Lung samples were collected for hybridization assay of I5NP concentrations at 6, 24 and 72 h post-end of dosing. The mean plasma concentration of I5NP at 15 minutes post-end of dose was 40.3±14.5 ng/mL. At 6 h post-end of dose, I5NP levels were 11.1 ng/mL (animal 103) and, at 24 h, 0.192 ng/mL (animal 104). The concentration of I5NP was less than the limit of quantitation (0.10 ng/mL) in plasma at 72 hours post-end of dosing (animal 105).

The relative concentrations of I5NP were measured in bronchiolar and peripheral lung tissues taken from the left cranial and right caudal lobes of the lungs. Relative concentrations were similar in the tissue samples taken at each time point and these declined with increasing time post-dose, although these were still detectible at 72 hours post-end of dosing.

Levels of siRNA in the lung tissues were at all times much higher than those in the systemic circulation. I5NP levels were very similar in all lobes of the lungs at each time point, post-dose, indicating that the test article had been delivered to all lobes. At approximately 30 minutes post-end of dosing, there appeared to be higher levels of I5NP in peripheral areas of the lung than in the bronchiolar regions, suggesting that test article, delivered to the bronchioles via an inhaled dose, was rapidly absorbed and distributed to the remainder of the lung tissue. In conclusion, following administration of single oro-nasal inhalation doses of a nebulised aerosol formulation of fluorescent-labeled REDD14Cy3.5 and/or I5NP siRNAs to male cynomolgus monkeys, the distribution in lungs and the systemic exposure (in plasma) of siRNA were determined. The highest mean concentrations of I5NP in plasma were observed at the first time point analysed post-dose indicating that absorption of I5NP into the systemic circulation from the inhaled dose was rapid. Concentrations in plasma were low and declined rapidly. Levels of I5NP in lungs were at all times greater than those in plasma and levels were still detectible at 72 h post-end of dosing. Distribution of both siRNAs in the lung was general and widespread. I5NP was found to be rapidly absorbed from the site of administration (bronchioles) and was found at high concentrations in the peripheral tissue of the lungs by 30 minutes post-end of dosing.

### Experimental Procedures

### Preparation of Dose Formulations

### Preparation of Treatment 1 and Treatment 3 Formulations

Each dose formulation was prepared on the day of dosing. Formulations were prepared under a laminar flow hood using clean techniques.

The I5NP siRNA was weighed out and dissolved in Sterile Water for Injection, USP in order to prepare a 40 mg/mL solution. All handling of the I5NP siRNA was performed protected from light or under yellow light conditions.

The resulting solution was filtered (0.22 μm filter) into a sterile container.

### Preparation of Treatment 2 Formulation

The dose formulation was prepared on the day of dosing. The formulation was prepared under a laminar flow hood using clean techniques. The I5NP siRNA was weighed out and dissolved in Sterile Water for Injection, USP in order to prepare a 40 mg/mL solution. The REDD14Cy3.5 siRNA was weighed out and dissolved in Sterile Water for Injection, USP in order to prepare a 40 mg/mL solution. All handling of the I5NP and REDD14Cy3.5 was performed protected from light or under yellow light conditions.

REDD14.Cy3.5 (40 mg/mL) and I5NP (40 mg/mL) solutions were mixed in a ratio of 1:9 and filtered (0.22 μm filter) into a sterile container.

### Pre-Treatment and Treatment Procedures

### Inhalation Exposure Equipment

Animals were treated with test aerosols using a pediatric anesthetic-type oronasal face mask fitted with inlet and outlet tubes and a breathing loop. During treatment, animals were placed in a restraint sling. The test atmospheres were generated using an Aeroneb (Aerogen) nebulizer, and assessed for output as described below. The level of exposure to the test article was achieved by varying the duration of exposure.

Prior to the start of treatment, animals were acclimated to the sling and exposure mask for gradually increasing periods of time. During the latter stages of acclimation, animals were exposed to an aerosol of 0.9% (w/v) sterile Sodium Chloride for Injection, USP to present them with all components of the exposure system.

### Pre-Study Atmosphere Characterization

Prior to the start of the treatment, atmosphere characterization of the test aerosols was performed using the I5NP siRNA formulation only.

For two Aerogen Aeroneb® Pro Laboratory nebulizer systems (one test and one back-up instrument), an evaluation of the test article output at the breathing zone was performed gravimetrically. Particle size distribution analysis of the article aerosols was performed through one mask, and the mass median aerodynamic diameter (MMAD)±geometric standard deviation (GSD) was calculated from the gravimetric data using a Cascade Impactor.

### Calculation of Achieved Dosage

The achieved dose of active ingredient (mg/kg/session) for each treatment level was determined as follows:

$$Achieved Dose of Active Ingredient ( mg / kg / day ) = \frac{\begin{matrix}\begin{matrix}RMV \times \\ Active Concentration \times \end{matrix} \\ T \times D\end{matrix}}{BW}$$

Where RMV (L/min)=respiratory minute volume measured twice during the pretreatment period*.
Active Concentration=aerosol concentration of active ingredient determined by (mgIL) gravimetric analysis during pretreatment period
T (min)=treatment time
D=total aerosol deposition fraction was assumed to be at 100%
BW (kg)=body weight per animal prior to treatment occasions * Measured during the pretreatment period using the Buxco Bio System XA

The theoretical inhalation achieved doses of siRNA were calculated to be as follows:

| | | | Gravimetric | Exposure | | Body | Theoretical |
|---|---|---|---|---|---|---|---|
| Treatment | Animal | RMV | concentration | duration | | weight | achieved dose |
| number | number | (L/min) | (mg/L)^{a} | (min) | Deposition | (kg) | (mg/kg/session) |
| 1 | 101 | 2.70 | 2.036 | 15 | 1 | 2.80 | 29.45 |
| | 102 | 2.66 | | 15 | | 2.60 | 31.24 |
| | 103 | 4.16 | | 10 | | 2.70 | 31.37 |
| | 106 | 5.58 | | 10 | | 3.60 | 31.56 |
| 2 | 101 | 2.70 | 2.036 | 15 | 1 | 2.80 | 29.45 |
| | 102 | 2.66 | | 13^{b} | | 2.80 | 25.14 |
| 3 | 103 | 4.16 | 2.036 | 10 | 1 | 2.90 | 29.21 |
| | 104 | 3.72 | | 10 | | 2.30 | 32.93 |
| | 105 | 4.53 | | 12 | | 3.80 | 29.13 |

^{a}Based on Pretreatment Results
^{b}Animal 102 received 13 minutes of a scheduled 15 minute exposure

### Respiratory Minute Volume Assessment

Twice during pretreatment, each monkey's respiratory minute volume was continuously recorded for a period of 15 minutes. The overall mean of the 15-minute interval was determined for each animal and it was assumed that this parameter was unaffected by treatment. Measurements were made using the Buxco Bio system XA.

### Treatment

Animals were treated as follows:

| | | Exposure | Target dose | |
|---|---|---|---|---|
| Treatment | Animal | duration | level | |
| number | number | (minutes) | (mg/kg/day) | Test article |
| 1 | 101, 102 | 15 | 35 | I5NP siRNA |
| | 103, 106 | 10 | | |
| 2 | 101, 102 | 15 | 35 | REDD14Cy3.5 |
| | | | | siRNA and I5NP |
| | | | | siRNA Ratio 1:9 |
| 3 | 103, 104 | 10 | 35 | I5NP siRNA |
| | 105 | 12 | | |

For Treatment 1

On Day 1 of the study, four of the available monkeys (101 to 104) were administered a target dose of 35 mg/kg of I5NP siRNA via an oronasal mask as a nebulized aerosol generated by the Aeroneb device. A 2.0 L/min dilution airflow was used. The total dose administered was calculated, based on the bodyweights and measured respiratory minute volumes of the animals, and on the achieved output of the Aeroneb device using the formulation concentration of 40 mg/mL. The administered dose was adjusted by changing the exposure times for the animals. Animals received treatment sequentially, rather than in parallel, using the single calibrated test Aeroneb.

### For Treatment 2

On Day 15 of the study, two of the available animals (101 and 102) were administered a target dose of 35 mg/kg of a mixture of I5NP and REDD14Cy3.5 (fluorescence-labelled) siRNA via an oronasal mask as a nebulized aerosol generated by the Aeroneb device. A 2.0 L/min dilution airflow was used. The total dose of siRNA administered was calculated, based on the bodyweights and measured respiratory minute volumes of the animals, and on the achieved output of the Aeroneb device using the formulation concentration of 40 mg/mL of siRNA. The administered dose was adjusted by changing the exposure times for the animals

### For Treatment 3

On Day 35 of the Study, the three remaining main study animals (103 to 105) were administered a target dose of 35 mg/kg of I5NP siRNA via an oronasal mask as a nebulized aerosol generated by the Aeroneb device. A 2.0 L/min dilution airflow was used. The total dose administered was calculated, based on the bodyweights and measured respiratory minute volumes of the animals, and on the achieved output of the Aeroneb device using the formulation concentration of 40 mg/mL. The administered dose was adjusted by changing the exposure times for the animals.

### Sample Collection for Analysis

### Blood/Plasma (Treatment 1)

A blood sample (0.5 mL) was collected at pre-dose, and at 15 minutes, 30 minutes, 1, 2, 4, 6, 8 and 24 hours post-end of inhalation, from the left or right femoral vein of each animal that received the full dose (101, 102, 103 and 106). Blood was collected into tubes with K₃EDTA as the anticoagulant and then placed on wet ice pending centrifugation.

### Blood/Plasma and Tissues (Treatment 2)

A blood sample (0.5 mL) was collected at pre-dose, at 15 minutes and at 30 minutes post-end of inhalation. Blood was collected into K₃EDTA tubes, from the left femoral vein of each animal (101 and 102), and placed on wet ice pending centrifugation.

As soon as possible following the 30-minute blood collection, the animals were given an intra-muscular injection of a pre-anesthetic agent (ketamine/xylazine cocktail) and transported to the necropsy area. The animals were then euthanized by an intravenous injection of sodium pentobarbital followed by exsanguination by incision of an axillary or femoral artery.

### Blood/Plasma and Tissues (Treatment 3)

A blood sample (0.5 mL) was collected at pre-dose and at 15 minutes post-end of inhalation from all animals (103, 104 and 105). A third blood sample was collected at 6 h (animal 103), 24 h (animal 104) or 72 h (animal 105) post-end of inhalation. Blood was collected, into K₃EDTA tubes, from the left or right femoral vein of each animal (103 to 105), and placed on wet ice pending centrifugation.

Immediately following the final blood collection, the animals were given an intra-muscular injection of a pre-anesthetic agent (ketamine/xylazine cocktail) and transported to the necropsy area. The animals were then euthanized by an intravenous injection of sodium pentobarbital followed by exsanguination by incision of an axillary or femoral artery. The following tissues and organs were collected from each animal: both lungs for hybridization assay, liver, kidneys (both), spleen, heart, brain and testes.

### Sample Processing

### Sample Processing for Blood/Plasma (Treatments 1, 2 and 3)

Blood was kept on wet ice until it was centrifuged, at 4° C. and 2700 rpm for approximately 10 minutes, to separate plasma (within 1 hour of collection). Plasma samples were separated, transferred to appropriately labeled vials and placed on dry ice prior to storage at approximately −80° C.

Plasma samples were tracked, using the Watson LIMS system.

### Sample Processing for Lungs (Treatment 2)

At the time of necropsy following Treatment 2, the lungs of animal 101 were removed from the animal. The lungs were separated into individual lobes. The bronchi/bronchioles and immediately surrounding regions of each lobe were dissected out as carefully as was practicable and pooled, per lobe. The remaining tissue areas were pooled, per lobe. Each pooled sample (i.e. bronchi/bronchioles and remaining tissue for each lobe) was stored deep-frozen (−80° C.) for analysis via hybridization assay. Lung samples from animal 101 were tracked using the Watson LIMS system.

At the time of necropsy following Treatment 2, the lungs, bronchi and trachea of animal 102 were removed from the animal. The lungs were perfused with 4% paraformaldehyde, introduced using a syringe, via the trachea and/or bronchus and stored in this fixative at ambient temperature for a period of approximately 21 hours. Following this, as much as possible of the original fixative fluid was withdrawn by syringe via the trachea/bronchus and replaced with 0.2% paraformaldehyde. The fixed lungs were analyzed for fluorescence distribution.

### Sample Processing for Kidneys (Treatment 2)

At the time of necropsy following Treatment 2, the right kidney of animal 102 was removed from the animal. The kidney was dissected into quarters by making one longitudinal and one transverse cut, each to pass through the hilus, and then immersed in 4% paraformaldehyde and refrigerated for approximately 21 hours. Following this, the fixing solution was changed to 0.2% paraformaldehyde and the kidney was analyzed for fluorescence distribution.

### Sample Processing for Lungs (Treatment 3)

At each specified time of necropsy following Treatment 3, the lungs of animals 103, 104 or 105 were removed from the animals. Each set of lungs were separated into individual lobes. The bronchi/bronchioles and immediately surrounding regions of each lobe were dissected out as carefully as was practicable and pooled, per lobe. The remaining tissue areas were pooled, per lobe. Each pooled sample (i.e. bronchi/bronchioles and remaining tissue for each lobe) was stored deep-frozen (−80° C.) for analysis via hybridization assay. Lung samples from animals 103, 104 and 105 were tracked using the Watson LIMS system.

### Sample Analysis

### Plasma and Lung Hybridization Analysis

Following Treatments 1 and 3, plasma samples were analyzed for I5NP siRNA concentration at PCS-MTL according to a validated hybridization method.

All lung samples from animal 101 (i.e. left cranial lobe, bronchioles and remainder; left caudal lobe, bronchioles and remainder; right cranial lobe, bronchioles and remainder; right middle lobe, bronchioles and remainder; right caudal lobe, bronchioles and remainder and right accessory lobe, bronchioles and remainder) were analyzed for I5NP siRNA concentration. For animals 103, 104 and 105, left cranial lobe (bronchioles and remainder) and right caudal lobe (bronchioles and remainder) were analyzed for I5NP siRNA concentration. Analysis was performed using a hybridization method and concentration values were extrapolated from a rat kidney calibration curve. The results obtained for lung samples were considered as semi-quantitative.

### Lung and Kidney Fluorescence Analysis

Following Treatment 2, lung and kidney samples from animal 102 were analyzed for distribution of fluorescence (from REDD14Cy3.5 siRNA). The results were considered to be qualitative.

### Data Analysis

### Plasma Concentration and Statistical Analysis

Mean plasma concentration values and standard deviations were calculated, where appropriate.

### Pharmacokinetics

Non-compartmental disposition kinetics in plasma was performed on concentration data from individual animals following Treatment 1. The following parameters were estimated as applicable:

| | |
|---|---|
| Cₘₐₓ | Highest observed plasma concentration. |
| tₘₐₓ | Time at which the highest concentration occurred. |
| k | Terminal rate constant determined by linear regression |
| | analysis of selected time points in the apparent |
| | terminal phase of the log plasma vs. time curves. |
| t_{1/2} | Terminal half-life calculated as ln2/k. |
| AUC₀₋ₜₗₐₛₜ | Area under the plasma concentration vs. |
| | time curve from 0 hours to the last quantifiable value. |
| AUC_{0-inf} | Area under the plasma concentration vs. |
| | time curve from 0 hours to infinity. |
| % Extrapolation | (AUC_{0-inf} − AUC₀₋ₜₗₐₛₜ)/ |
| AUC_{0-inf} | AUC_{0-inf} × 100 |

All pharmacokinetic parameters were estimated using WinNonlin version 3.2 computer software.

### Results

### Animal and Dose Characteristics

The mean body weight of the male monkeys on the day prior to dosing Treatment 1 was 2.9±0.46 kg (range 2.6 kg to 3.6 kg). For Treatment 2 the bodyweight of animals 101 and 102 was 2.8 kg for each animal, whilst for Treatment 3, the mean bodyweight was 3.0±0.76 kg (range 2.3 kg to 3.8 kg).

Dose exposure times and estimated dose levels of siRNA administered: for Treatment 1, dose exposure times were 15 minutes (101, 102) or 10 minutes (103, 106). The mean calculated administered dose was 30.9±0.98 mg/kg (range 29.45 to 31.56 mg/kg). For Treatment 2, animal 101 received a calculated dose of 29.45 mg/kg over 15 minutes and animal 102 a calculated dose of 25.14 mg/kg over 13 minutes. For Treatment 3, dose exposure times were 10 minutes (103, 104) or 12 minutes (105). The mean calculated administered dose was 30.4±2.17 mg/kg (range 29.13 to 32.93 mg/kg).

### Clinical Observations

No treatment-related clinical signs were observed in any of the monkeys following inhalation doses of I5NP or a mixture of I5NP and REDD14Cy3.5 siRNAs. Miscellaneous macroscopic findings seen in various organs and tissues were considered to be related to sexual immaturity, agonal or incidental, and to be of no toxicological significance.

Concentration and Pharmacokinetics of I5NP siRNA in Plasma
I5NP siRNA Concentrations in Plasma

Following Treatment 1, the highest mean concentration of I5NP in plasma of male monkeys was 21.0±5.01 ng/mL, observed at 15 minutes following the end of dosing (the first time point measured). Thereafter, plasma I5NP levels declined until 8 hours post-end of dose (0.588±0.561 ng/mL, approximately 3% of the Cₘₐₓ value). In animal 101, I5NP remained detectible at 24 h post-end of dosing (0.205 ng/mL) but values were below the limit of quantification (LLOQ) at this time point in all other animals. The LLOQ for the assay was 0.10 ng/mL.

Following Treatment 3, blood samples were collected from all animals at 15 minutes post-end of dose and terminally from a single animal per time point (6 h, 14 h and 72 h post-end of dose). The mean plasma concentration of I5NP at 15 minutes post-end of dose was 40.3±14.5 ng/mL. At 6 h post-end of dose, I5NP levels were 11.1 ng/mL (animal 103) and, at 24 h, 0.192 ng/mL (animal 104). The concentration of I5NP was less than the limit of quantitation (0.10 ng/mL) in plasma at 72 hours post-end of dosing (animal 105).

Pharmacokinetics of I5NP siRNA in Plasma (Treatment 1)

The mean value for AUC₀₋ₜₗₐₛₜ was 38.9±23.6 ng·h/mL (range 15.8 to 70.3 ng·h/mL). The elimination phase was considered to be well-defined for all four animals and thus the elimination rate constant (kₑₗ), half-life of elimination (t_{1/2}) and AUC_{0-inf} were estimated for each monkey. The mean kₑₗ was 0.279±0.143 h⁻¹, the mean t_{1/2} was 3.12±1.83 h and the mean AUC_{0-inf} was 40.6±24.5 ng·h/mL (range 16.1 to 72.0 ng·h/mL). The % extrapolation from tₗₐₛₜ to infinity ranged from 0.82 to 9.40%.

Qualitative Distribution of REDD14Cy3.5 siRNA in Lung and Kidney

The fluorescent label was detected in all sections taken from all lobes of the lungs. Fluorescent label was observed radiating from the bronchioles outward into the alveolar walls. The strongest signals appeared to be associated with the right caudal lobe and the caudal section of the left cranial lobe.

There was little label detected in the right kidney with, at most, a slight residual staining being observed in the lumen of the kidney tubules.

Semi-Quantitative Distribution of I5NP siRNA in Lung

For lung tissues from animal 101 (following Treatment 2), each lobe was separated into the bronchioles and immediately surrounding tissue and the remainder, peripheral lobe tissue. Measurements were made in both tissue types, and for all 6 lobes of the lung. At approximately 30 minutes post-end of dosing, I5NP was found in high concentrations in both bronchioles and in peripheral tissues of all lobes of each lung. Concentrations were similar throughout all the lobes and were generally higher in the peripheral tissues than in the bronchiolar samples. Following these results, animals 103, 104 and 105 received an inhalation dose of I5NP and the relative concentrations were measured in bronchiolar and peripheral lung tissues taken from the left cranial and right caudal lobes of the lungs at 6, 24 and 72 hours post-end of dosing. Relative concentrations of I5NP were similar in the tissue samples taken at each time point. Concentrations of I5NP declined with increasing time post-dose, but were still detectible at 72 hours post-end of dosing.

### Discussion

The highest mean concentrations of I5NP in plasma were observed at the first time point analysed post-dose (15 minutes post-end of dosing), indicating that absorption of I5NP into the systemic circulation from the inhaled dose was rapid. Concentrations in plasma were low and declined rapidly up to 8 h or 24 h post-end of dosing, after when they were below the limit of quantitation. There was a considerable degree of variation between individual animals, as demonstrated by the variation in the half-life of elimination (i.e. from 1.5 to 5.7 h) and the AUC_{0-inf} (range 16.1 to 72.0 ng·h/mL).

Levels of siRNA in the lung tissues were much higher than in the systemic circulation. I5NP levels were very similar in all lobes of the lungs at each time point, post-dose, indicating that the test article had been delivered to all lobes. At approximately 30 minutes post-end of dosing, there appeared to be higher levels of I5NP in peripheral areas of the lung than in the bronchiolar regions, suggesting that test article, delivered to the bronchioles via an inhaled dose, was rapidly absorbed and distributed to the remainder of the lung tissue. Levels of 15NP in lung tissues declined with increasing time post dose but were still detectible at 72 h post-end of dosing. At all times, I5NP levels in lungs were higher than in plasma.

A comparison of the distribution of I5NP and REDD14Cy3.5 siRNA, the latter analysed qualitatively via fluorescence detection, indicated that there was little difference in the distribution of the two test articles in lung.

### CONCLUSION

Following administration of single oronasal inhalation doses of a nebulised aerosol formulation of fluorescent-labelled REDD14Cy3.5 and/or I5NP siRNAs to male cynomolgus monkeys, the distribution in lungs and the systemic exposure (in plasma) of siRNA were determined.

The highest mean concentrations of I5NP in plasma were observed at the first time point analysed post-dose indicating that absorption of I5NP into the systemic circulation from the inhaled dose was rapid. Concentrations in plasma were low and declined rapidly. Levels of I5NP in lungs were at all times greater than those in plasma, and levels were still detectible at 72 h post-end of dosing. Distribution of both siRNAs in the lung was general and widespread. I5NP was found to be rapidly absorbed from the site of administration (bronchioles) and was found at high concentrations in the peripheral tissue of the lungs by 30 minutes post-end of dosing.

Modified siRNAs (formulated in aqueous solution) which are directed against RTP801, p53, TP53BP2, LRDD, CYBA, ATF3, CASP2, NOX3, HRK, C1QBP, BNIP3, MAPK8, MAPK14, Rac1, GSK3B, P2RX7, TRPM2, PARG, CD38, STEAP4, BMP2, GJA1, TYROBP, CTGF, SPP1, RTN4R, ANXA2, RHOA and DUOX1 are tested essentially as presented above for the two described siRNAs, and similar results are obtained showing that the siRNAs are delivered successfully to the lung.

### Example 3

### Effect of Nrf2 siRNA Pulmonary Administration on Tumor Growth In Vivo

### Methods:

Tumor Xenografts: A549 cells (5×10⁶) were injected into the hind leg of male athymic nude mice and the tumor was measured weekly. The tumor volumes were measured using the following formula: [length (mm)×width (mm)×width (mm)×0.52]. In the lung metastasis experiments, 2×106 A549-C8-luc cells were injected into SCID-Beige mice (Charles River, Mass.) intravenously.

For in vivo experiments, all siRNA compounds were chemically synthesized being stabilized by alternating 2-O′-Me modifications on both strands. The sequence of siRNA targeting human Nrf2 used for in vivo experiments is 5′-UCCCGUUUGUAGAUGACAA-3′ (sense) and 5′-UUGUCAUCUACAAACGGGA-3′ (antisense). The sequence of control siRNA targeting GFP is 5′-GGCUACGUCCAGGAGCGCACC-3′ (sense) and 5′-GGUGCGCUCCUGGACGUAGCC-3′ (antisense).

For lung tumor delivery, female C57B6 mice were injected with Lewis Lung Carcinoma (LLC) cells (0.5×10⁶) intravenously, 24 days prior to the delivery experiment. Upon development of lung metastases, mice were administered with 100 μg/mouse of Cy3-labeled naked chemically stabilized reference siRNA via nebulizer inhalation on 3 consequent days. Mice were euthanized 24 hrs after the last inhalation. Upon termination, lungs were inflated with ice-cold 4% paraformaldehyde, followed by manual sectioning with razor blades. Clearly visible large surface tumors were sectioned separately. Resulting sections were analyzed by Bio-Rad Confocal microscope using a 20× Water objective and 2× zoom combined to give a total of 40× magnification. Control, non-siRNA-treated lungs were used to set up background fluorescence level.

For aerosol delivery of Nrf2 or GFP siRNA into lung tumors, 100 μg of siRNA duplex diluted in PBS was aerosolized using a nebulizer. Mice were given three dose of siRNA (100 μg/dose) every week, for 4 weeks, using a nebulizer. Intraperitoneal injections of carboplatin were given at a dose of 30 mg/kg body weight twice/week.

In Vivo Imaging: Animals inoculated with A549-C8-luc cells, which express a luciferase reporter gene, were anesthetized and injected intraperitoneally with 250 ul of luminescent substrate (15 mg/ml stock) D-Luciferin Firefly (Xenogen Cat# XR-1001). The animals were then imaged and analyzed by using the Xenogen IVIS Optical Imaging Device in the Johns Hopkins Oncology Center.

Statistical Analysis-Statistical comparisons were performed by Student's t-tests. A value of p<0.05 was considered statistically significant. Tumor weights and changes in tumor volume were summarized using descriptive statistics. Differences in tumor measures between treatment groups were examined using linear regression models with generalized estimating equations (GEE). The distributions of both tumor measurements were skewed, so log transformations were used.

To demonstrate uptake of siRNA by lung tumors, Cy3 labeled RTP801 siRNA (REDD14—see above) was delivered into mice bearing lung tumors using a nebulizer. Small intra-parenchymal tumors revealed a robust Cy3 signal. The large surface-protruding tumors also showed Cy3 signal but the intensity was several folds lower.

The delivery of naked siRNA duplexes into orthotopic lung tumors was examined. Mice were injected with Lewis lung carcinoma cells and 24 days later (when the mice developed larger tumors) mice were inhaled for three consecutive days with 100 μg/day/mouse of Cy3 labeled naked chemically stabilized reference siRNA using a nebulizer. Twenty four hours after last siRNA administration, mice were sacrificed; the lungs were harvested and sectioned. Resulting sections were analyzed by Bio-Rad Confocal microscope using a 20× Water objective and 2× zoom combined to give a total of 40× magnification. Control, non-siRNA-treated lungs were used to set up background fluorescence level. The fluorescence results revealed localization of Cy3 labeled siRNA in a large surface tumor, especially in intraparenchymal tumor. The large surface-protruding tumors showed Cy3 signal but the intensity was several folds lower than that observed in the small intra-parenchymal tumors.

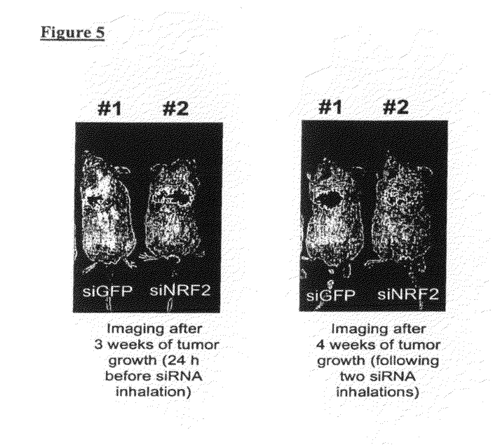

In the next step, mice with orthotopic growing tumors expressing ARE-Luc Nrf2-dependent reporter were used. The mice with A549 lung tumors were treated with Nrf2 siRNA intranasally using a nebulizer followed by carboplatin treatment. As demonstrated in FIG. 5, Nrf2 siRNA administered intranasally inhibited Nrf2 reporter activity in vivo, indicating specific inhibition of Nrf2 expression using Nrf2 siRNA following intranasal administration. Mice receiving Nrf2 siRNA together with carboplatin demonstrated higher growth inhibition as compared with control mice receiving GFP siRNA together with carboplatin (Table 3.1). The mean lung weight at termination and the luminescent flux intensity (evaluated by in vivo imaging) were lowest in mice treated with Nrf2 siRNA+ carboplatin. Thus, combination of Nrf2 siRNA with carboplatin/radiation led to a reduction in tumor growth after 4 weeks of treatment as compared to either agent alone.

TABLE 3.1
Mean (SD) of lung tumor weights following intranasal treatment
with Nrf2 siRNA alone, and in combination with carboplatin.

| Treatment | Mean (SD) tumor weight (mg) |
|---|---|
| GFP siRNA | 710 (167.93) |
| GFP siRNA + carboplatin | 480 (95.13) |
| Nrf2 siRNA | 802 (172.25) |
| Nrf2 siRNA + carboplatin | 370 (62.05) |
| Vehicle siRNA | 760 (98.99) |
| Vehicle siRNA + carboplatin | 495 (49.5) |

Further information is included in PCT application No. PCT/IL2008/000391, assigned to the assignee of the instant application, which is hereby incorporated by reference in its entirety.

Additional siRNA molecules directed towards any of the genes disclosed herein are tested in a similar manner, in which it is shown that they have a similar effect and inhibit the growth of lung tumors.

1-14. (canceled)

15. A method for treating a subject suffering from a lung disease which comprises administering to the subject's inner lung a modified siRNA compound in the form of an aerosol and in an amount effective to inhibit expression of a target gene associated with the lung disease in the inner lung of the subject so as to thereby treat the subject.

16. The method of claim 15, wherein the siRNA compound is 23 nucleotides or less in length.

17. The method of claim 16, wherein the siRNA compound is 19-23 nucleotides in length.

18. The method of claim 15, wherein the compound is administered at a dose in the range 0.1 to 10 grams per kilogram of body weight of the subject per day.

19. The method of claim 15, wherein the lung disease is selected from the group consisting of chronic obstructive pulmonary disease (COPD), lung cancer, asthma, cystic fibrosis, emphysema, bronchitis, bronchiectasis, interstitial lung disease, pneumonia, Acute Lung Injury, and Acute Respiratory Distress Syndrome.

20. The method of claim 15, wherein the aerosol comprises particles having an average particle size of about 1-5 micrometers in diameter.

21. The method of claim 20, wherein the aerosol comprises particles having an average particle size of about 4.1-4.9 micrometers in diameter.

22. The method of claim 21, wherein the aerosol comprises particles having an average particle size of 4.4-4.7 micrometers in diameter.

23. The method of claim 22, wherein the aerosol comprises particles having an average particle size of 4.5-4.6 micrometers in diameter.

24. The method of claim 15, wherein the aerosol is administered by nasal administration.

25. The method of claim 15, wherein the aerosol is administered by oronasal administration.

26. The method of claim 15, wherein the modified siRNA compound comprises a modification to a sugar moiety, a modification to a base moiety, and/or a modification to a linkage between two ribonucleotides.

27. The method of claim 26, wherein the modified siRNA compound comprises a modification to a sugar moiety.

28. The method of claim 27, wherein the modified siRNA compound comprises a 2′OMe modified sugar moiety.

29. The method of claim 15, wherein the modified siRNA compound is a naked siRNA compound.
